SEQUENCE LISTING (9 sequences)

SEQ ID NO: 1 - protein, 15 aa, synthetic construct
GLSDEWQQVLNVWGK

SEQ ID NO: 2 - protein, 15 aa, synthetic construct
VEADIAGHGQEVLIR

SEQ ID NO: 3 - protein, 12 aa, synthetic construct
TEAEMKASEDLK

SEQ ID NO: 4 - protein, 14 aa, synthetic construct
HGTVVLTALGGILK

SEQ ID NO: 5 - protein, 17 aa, synthetic construct
GHHEAELKPLAQSHATK

SEQ ID NO: 6 - protein, 20 aa, synthetic construct
IPIKYLEFISDAIIHVLHSK

SEQ ID NO: 7 - protein, 15 aa, synthetic construct
HPGDFGADAQGAMTK

SEQ ID NO: 8 - protein, 12 aa, synthetic construct
ALELFRNDIAAK

SEQ ID NO: 9 - protein, 8 aa, synthetic construct
YKELGFQG

(19) United States
(12) Patent Application Publication
(10) Pub. No.: US 2024/0400609 A1
(43) Pub. Date: Dec. 5, 2024

(54) COMPOSITIONS AND METHODS FOR MULTIPLEXED PLIMB-MEDIATED RADICAL LABELING

(71) Applicant: Immuto Scientific, Madison, WI (US)

(72) Inventors: Samantha Knott, Fitchburg, WI (US); Daniel Benjamin, Madison, WI (US); Faraz Choudhury, Fitchburg, WI (US); James Dowell, Middleton, WI (US)

(21) Appl. No.: 18/679,737

(22) Filed: May 31, 2024

Related U.S. Application Data
(60) Provisional application No. 63/470,704, filed on Jun. 2, 2023.

(51) Int. Cl. C07K 1/13; G01N 33/58; G01N 33/68

(52) U.S. Cl. CPC C07K 1/13; G01N 33/58; G01N 33/6848

(57) ABSTRACT

Provided herein are materials and methods for plasma-based multiplexed labeling of biological molecules. In some aspects, provided herein is an optimized method for simultaneous labeling of proteins with trifluoromethyl, nitro, and hydroxyl radicals generated by a plasma in a single reaction vial, such as for protein footprinting.

### STATEMENT OF RELATED APPLICATIONS

This application claims the benefit of U.S. Provisional Patent Application No. 63/470,704, filed Jun. 2, 2023, the entire contents of which are incorporated herein by reference for all purposes.

### STATEMENT REGARDING FEDERAL FUNDING

This invention was made with government support under 2R44GM134849-01 and 2R44GM134849-02 awarded by the National Institutes of Health. The government has certain rights in the invention.

### SEQUENCE LISTING

The text of the computer readable sequence listing filed herewith, titled “IMMUT-42093-202_SQL”, created Aug. 21, 2024, having a file size of 8,919 bytes, is hereby incorporated by reference in its entirety.

### TECHNICAL FIELD

Provided herein are materials and methods for multiplexed labeling of biological molecules. In some aspects, provided herein is a method for simultaneous labeling of proteins with trifluoromethyl, nitro, and hydroxyl radicals in a single reaction vial, such as for protein footprinting.

### BACKGROUND

Chemical labeling of proteins coupled to mass spectrometry (MS), such as hydrogen-deuterium exchange (HDX) and radical footprinting, are a family of techniques which have been fundamental for the understanding of protein higher order structure (HOS) and the field of structural biology. The large sample requirements, protein size limitations and time for crystallization of high-resolution techniques, such as cryogenic electron microscopy (cryo-EM) and x-ray crystallography, often make highly dynamic protein samples less amenable to study. Although footprinting cannot provide the atomic resolution of crystallography, it is considerably higher throughput. Additionally, the resolution from a footprinting experiment approaches single amino acid level and rivals throughput of techniques such as peptide scanning or epitope binning, which are often limited to the peptide level.

The most popular and frequently applied protein footprinting technique is Hydrogen Deuterium Exchange (HDX), which is based on the principle of protein backbone amide proton exchange and labeling with deuterium. Despite its popularity, HDX-MS is hindered by the inherent shortcoming of back exchange and the reversibility of deuterium labeling, which can result in a significant loss of signal. Additionally, the resulting data is generally limited to the peptide level resolution. Radical based protein footprinting provides an attractive alternative to HDX, as the chemical moieties are covalently and irreversibly bound. Chemical labeling utilizing radical species can be introduced in several ways for protein footprinting and can yield both peptide and residue level information. The leading footprinting approach is Hydroxyl Radical Protein Footprinting (HRF), where hydroxyl (•OH) radicals are generated in solution. The OH radicals can be generated by several different methods including synchrotron radiation or laser photolysis of hydrogen peroxide (H₂O₂), otherwise known as fast-photochemical oxidation of proteins (FPOP).

Although HRF is a powerful technique for analysis of protein structure, motion, and interactions, it can be limited by resolution. Due to kinetics and reactivity rates, OH radicals typically only routinely label 6 out of the 20 amino acids: methionine (M), cystine (C), tryptophan (W), phenylalanine (F), histidine (H) and tyrosine (Y). Considering these residues may only cover ˜5-30% of the protein sequence, the resolution of the data from hydroxyl radical footprinting (HRF) experiments is limited by the ability to acquire information on regions containing these six residues. Accordingly, what is needed are improved methods for labeling of biological molecules, such as protein footprinting, that effectively label all amino acids with high resolution and high throughput.

### SUMMARY

In some aspects, provided herein are plasma-based methods for multiplexed labeling a biological molecule. In some embodiments, provided herein are methods for multiplexed labeling of a biological molecule with trifluoromethyl and hydroxyl and/or nitro radicals, comprising providing a sample comprising two or more radical precursors and a biological molecule; and generating a plasma in the sample, thereby converting the two or more radical precursors in the sample into trifluoromethyl and hydroxyl and/or nitro radicals which interact with the biological molecule, thereby labeling the biological molecule. In some embodiments, the two or more radical precursors comprise sodium triflinate and hydrogen peroxide. In some embodiments, the two or more radical precursors comprise sodium triflinate, hydrogen peroxide, and air. In some embodiments, the two or more radical precursors comprise sodium triflinate and tert-butyl hydroperoxide (TBHP).

In some embodiments, the sample additionally comprises phosphate buffered saline (PBS) or HEPES buffer. In some embodiments, the plasma is generated for 1 ns to 72 hours. In some embodiments, plasma is generated from a plasma source point of a plasma electrode. In some embodiments, the plasma source point is placed within the sample. In some embodiments, the plasma source point is placed at an interface of a gas and the sample. In some embodiments, the plasma source point is placed at a distance of 10 cm or less from the sample.

In some embodiments, the method further comprises quenching the sample with a solution comprising a radical quencher following generating the plasma in the sample. In some embodiments, the radical quencher is methionine, cysteine, homocysteine, or taurine.

In some embodiments, the sample comprises one or more radical scavengers.

In some embodiments, the biological molecule comprises a protein. In some embodiments, the protein has been treated with one or more cleavage factors. In some embodiments, the method further comprises identifying labeling of the biological molecule with the trifluoromethyl and hydroxyl and/or nitro radicals using mass spectrometry.

In some embodiments, provided herein is a method for multiplexed labeling a biological molecule, comprising providing a sample comprising a first radical precursor and a biological molecule and generating a plasma in the sample, thereby converting the first radical precursor in the sample into a first radical which interacts with the biological molecule, thereby labeling the biological molecule with the first radical. In some embodiments, the method further comprises adding a second radical precursor to the biological sample and generating a plasma in the sample, thereby converting the second radical precursor in the sample into a second radical which interacts with the biological molecule, thereby labeling the biological molecule with the second radical. In some embodiments, the first radical precursor is sodium triflinate and the second radical precursor is hydrogen peroxide or tert-butyl hydroperoxide (TBHP). In some embodiments, the first radical precursor is hydrogen peroxide or tert-butyl hydroperoxide (TBHP) and the second radical precursor is sodium triflinate.

In some embodiments, provided herein is a method for multiplexed labeling a biological molecule, the method comprising providing a sample comprising a first radical precursor, a second radical precursor, and a biological molecule; generating a first plasma in the sample, thereby converting the first radical precursor in the sample into a first radical which interacts with the biological molecule, thereby labeling the biological molecule with the first radical; and generating a second plasma in the sample, thereby converting the second radical precursor in the sample into a second radical which interacts with the biological molecule, thereby labeling the biological molecule with the second radical. In some embodiments, the first radical precursor is sodium triflinate and the second radical precursor is hydrogen peroxide or tert-butyl hydroperoxide (TBHP). In some embodiments, the first radical precursor is hydrogen peroxide or tert-butyl hydroperoxide (TBHP) and the second radical precursor is sodium triflinate.

In some embodiments, each plasma is generated for 1 ns to 72 hours. In some embodiments, plasma is generated from a plasma source point of a plasma electrode. In some embodiments, the plasma source point is placed within the sample. In some embodiments, the plasma source point is placed at an interface of a gas and the sample. In some embodiments, the plasma source point is placed at a distance of 10 cm or less from the sample.

In some embodiments, the method further comprises comprising quenching the sample with a solution comprising a radical quencher following generating the first plasma and/or the second plasma. In some embodiments, the radical quencher is methionine, cysteine, homocysteine, or taurine.

In some embodiments, the sample comprises one or more radical scavengers.

In some embodiments, the biological molecule comprises a protein. In some embodiments, the protein has been treated with one or more cleavage factors. In some embodiments, the method further comprises identifying labeling of the biological molecule with the trifluoromethyl and hydroxyl and/or nitro radicals using mass spectrometry.

In some aspects, provided herein are kits. In some embodiments, provided herein is a kit for use in a method described herein, the kit comprising one or more radical precursors, one or more buffers, and/or one or more quenchers. In some embodiments, the kit comprises sodium triflinate and/or hydrogen peroxide, and a buffer selected from PBS and HEPES buffer. In some embodiments, the kit comprises sodium triflinate and/or tert-butyl hydroperoxide (TBHP), and comprising a buffer selected from PBS and HEPES buffer.

### BRIEF DESCRIPTION OF THE DRAWINGS

FIG. 1 shows an exemplary workflow for one-pot dual PLIMB-mediated hydroxyl (•OH), Nitro (•NO2) and trifluoromethyl (•CF3) protein-footprinting.

FIG. 2 shows a Venn diagram comparing residue labeling coverage of myoglobin in (•OH) footprinting only vs. PLIMB-mediated hydroxyl (•OH), Nitro (•NO2), and trifluoromethyl (•CF3) radical protein-footprinting. All residues were confirmed by MS/MS. Overlapping regions indicate residues reactive to multiple radical species.

FIG. 3 shows an exemplary workflow for complementary PLIMB-mediated OH, NO2 and CF3 radical footprinting for epitope mapping. 1) Solution of antigen alone and in complex with antibody solubilized in optimized labeling buffer conditions (PBS, pH 7.4, 10 mM 11202 and 50 mM sodium triflinate). 2) PLIMB-mediated radical labeling. 3) Digestion and LC-MS/MS 4) Data analysis, validation and % modification of peptides calculated for antigen alone and in complex. 5) Peptides showing protection mapped to corresponding crystal structure.

FIG. 4 shows HER2 coverage of PLIMB-mediated OH radical vs. dual OH, NO2 and CF3 radical footprinting. Residues modified, overall percentage of residues modified and the average length between modifications observed over the entire protein sequence observed.

FIGS. 5A-D show MS/MS peptide spectra and localization of both the unmodified and PLIMB mediated radical modification of HER2. FIG. 5A shows MS/MS of the NO2 modification localized to tryptophan (W). FIG. 5B shows MS/MS of the OH modification localized to tryptophan (W). FIG. 5C shows MS/MS of the CF3 modification localized to tryptophan (W). FIG. 5D shows MS/MS of the unmodified peptide.

FIGS. 6A-6B show epitope mapping of the HER2/Herceptin complex with PLIMB-mediated OH radical footprinting. FIG. 6A shows the known crystal structure of HER2/Herceptin complex with overlapping PLIMB epitope determination. FIG. 6B shows the percent (%) OH radical modification of HER2/Herceptin epitope peptides after PLIMB-mediated footprinting.

FIG. 7A shows a comparison of residue level PLIMB-mediated OH radical vs. dual PLIMB-mediated OH, NO2 and CF3 radical footprinting of HER2/Herceptin epitope region 1.

FIG. 7B shows a comparison of residue level PLIMB-mediated OH radical vs. dual PLIMB-mediated OH, NO2 and CF3 radical footprinting of HER2/Herceptin epitope region 2.

FIGS. 8 A-B show sub-residue level resolution of PLIMB-mediated trifluoromethyl (•CF3) labeling of TNFα. FIG. 8A shows a TNFα peptide modified on phenylalanine (F) by CF3 and the possible modified/substituted products (ortho, meta, para). FIG. 8B shows the extracted ion chromatogram elution of both the unmodified and CF3 modified (F) peptide, with each circle representing a targeted MS/MS event over the multiple eluting peaks.

FIGS. 9A-C shows optimization of radical dose for simultaneous PLIMB-mediated hydroxyl (•OH), Nitro (•NO2) and trifluoromethyl (•CF3) labeling of protein. FIG. 9A shows the percent (%) OH modification of peptides across lysozyme calculated for the different PLIMB doses (10, 60, 75, 90 and 120 seconds) in 10 mM H2O2, 50 mM H2O2. FIG. 9B shows the percent (%) CF3 modification of peptides across lysozyme calculated for the different PLIMB doses (10, 60, 75, 90 and 120 seconds) in 10 mM H2O2, 50 mM H2O2. FIG. 9C shows the percent (%) NO2 modification of peptides across lysozyme calculated for the different PLIMB doses (10, 60, 75, 90 and 120 seconds) in 10 mM H2O2, 50 mM H2O2.

FIG. 10 shows PLIMB-mediated trifluoromethyl (•CF3) labeling of lysosome in varied sodium triflinate concentrations. The percent (%) CF3 modification of lysozyme peptides was calculated for solutions utilizing different sodium triflinate concentrations (50 uM, 10 mM, 50 mM, 150 mM and 500 mM) in 10 mM H2O2, PBS (pH 7.4) and treated with a fixed 75-second PLIMB dose.

FIG. 11 shows PLIMB-mediated trifluoromethyl (•CF3) labeling of lysosome in PBS vs HEPES buffer. The percent (%) CF3 modification of peptides across lysozyme in 50 mM CF3 and 10 mM H202 was calculated for the different experimental buffer conditions: 5 mM PBS, pH 7.4 or 5 mM HEPES, pH 6.0.

FIG. 12 shows optimization of hydrogen peroxide concentration for simultaneous PLIMB-mediated hydroxyl (•OH), Nitro (•NO2) and trifluoromethyl (•CF3) footprinting of protein. The percent (%) CF3 modification of peptides across lysozyme was calculated for the different experimental conditions: 0, 10, 50, 100 mM H2O2 and 50 mM CF3 for all.

FIG. 13 shows optimization of TBHP for simultaneous PLIMB-mediated hydroxyl (•OH), Nitro (•NO2) and trifluoromethyl (•CF3) footprinting of protein. The percent (%) CF3 modification of peptides across lysozyme was calculated for the different experimental conditions: 0, 1, 5, 10, 50, and 100 mM H2O2 or TBHP.

FIG. 14 shows optimization of electrode position for dual, simultaneous PLIMB-mediated hydroxyl (•OH). Nitro (•NO2) and trifluoromethyl (•CF3) labeling of protein in the presence of radical scavengers. The percent (%) modification of peptides across lysozyme was calculated for samples in 10 mM H2O2, 50 mM H2O2 and subjected to a fixed PLIMB dose with the electrode submerged in-solution or suspended above the solution.

FIG. 15 shows optimization of PLIMB-mediated hydroxyl (•OH) labeling of myoglobin [2-97] in the presence and absence of the radical scavenger DMSO. After performing hydroxyl radical protein footprinting with PLIMB in the presence and absence of DMSO, percent modification of peptides across the entire myoglobin sequence was calculated for the different experimental conditions: 0, 0.5, 1.0, 1.5 and 2.0% DMSO (v/v). T-tests were performed to detect statistically significant differences in modification between conditions. Asterisks indicate a significant decrease in percent modification across conditions (p<0.05).

FIG. 16 shows optimization of PLIMB-mediated hydroxyl (•OH) labeling of myoglobin [100-154] in the presence and absence of the radical scavenger DMSO. After performing hydroxyl radical protein footprinting with PLIMB in the presence and absence of DMSO, percent modification of peptides across the entire myoglobin sequence was calculated for the different experimental conditions: 0, 0.5, 1.0, 1.5 and 2.0% DMSO (V/V). T-tests were performed to detect statistically significant differences in modification between conditions. Asterisks indicate a significant decrease in percent modification across conditions (p<0.05).

FIG. 17 shows optimization 1 of pH conditions for dual, simultaneous PLIMB-mediated hydroxyl (•OH). Nitro (•NO2) and trifluoromethyl (•CF3) labeling of protein. 50 uL aliquots of solution were prepared and subjected to various PLIMB doses and the resulting pH is shown above.

FIG. 18 shows optimization 2 of pH conditions for dual, simultaneous PLIMB-mediated hydroxyl (•OH), Nitro (•NO2) and trifluoromethyl (•CF3) labeling of protein. 50 uL aliquots of solution were prepared and subjected to various PLIMB doses and the resulting pH is shown above.

FIG. 19 shows optimization 3 of pH conditions for dual, simultaneous PLIMB-mediated hydroxyl (•OH), Nitro (•NO2) and trifluoromethyl (•CF3) labeling of protein. 50 uL aliquots of solution were prepared and subjected to various PLIMB doses and the resulting pH is shown above.

FIG. 20 shows optimization 4 of pH conditions for dual, simultaneous PLIMB-mediated hydroxyl (•OH), Nitro (•NO2) and trifluoromethyl (•CF3) labeling of protein. 50 uL aliquots of solution were prepared and subjected to various PLIMB doses and the resulting pH is shown above.

FIGS. 21A-21B show PLIMB-mediated trifluoromethyl (•CF3) labeling of lysosome in the presence or absence of transition metal catalysts. All solutions were prepared in PBS, pH 7.4 with 50 mM of the sodium triflinate additive. After PLIMB treatment in the presence and absence of Cu (II) or Fe (II), percent CF3 modification of peptides across the entire lysozyme sequence was calculated for the different experimental conditions. FIG. 21A shows a comparison between 50 mM sodium triflinate in the presence or absence of transition metal catalysts (Cu II and Fe II). FIG. 21B shows a comparison between the baseline trifluoromethyl footprinting conditions (10 mM H2O2, 50 mM sodium triflinate) in the presence and absence of transition metal catalysts (Cu II and Fe II).

### DETAILED DESCRIPTION

Section headings as used in this section and the entire disclosure herein are merely for organizational purposes and are not intended to be limiting.

All publications, patent applications, patents and other references mentioned herein are incorporated by reference in their entirety.

### 1. Definitions

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

### 2. Methods for Labeling of Biological Molecules

Radical based “protein footprinting” provides a healthy balance between throughput and resolution. Protein footprinting approaches probe changes in structure by mapping solvent-accessible surface area (SASA) via labeling of amino acid sidechains in solution with a chemical moiety, followed by quantification using MS. Applications of protein footprinting include antibody epitope localization, where regions of protein interactions are characterized by changes in SASA between two different conditions (e.g., bound and unbound to the antigen's corresponding antibody). A region showing protection, or a decreased level of labeling in the bound condition versus unbound, can therefore by an indication a binding region.

An exemplary footprinting approach is Hydroxyl Radical Protein Footprinting (HRF), where hydroxyl (•OH) radicals are generated in solution. The OH radicals can be generated by several different methods including synchrotron radiation or laser photolysis of hydrogen peroxide (H₂O₂), otherwise known as fast-photochemical oxidation of proteins (FPOP). Recently, several of the inventors herein developed a technique for generation of radicals referred to as Plasma Induced Modification of Biomolecules (PLIMB) Minkoff, B. B., et al., (2017). Scientific Reports, 7(1)), which utilizes an electrode with an applied potential and high energy plasma to generate microsecond-timescale bursts of hydroxyl radicals generated from water. Although HRF is a powerful technique for analysis of protein structure, motion, and interactions, it can be limited by resolution. OH radicals typically only routinely label 6 out of the 20 amino acids: methionine (M), Cystine©, tryptophan (W), phenylalanine (F), histidine (H) and tyrosine (Y). Considering these residues may only cover ˜5-30% of the protein sequence, the resolution of the data from hydroxyl radical footprinting (HRF) experiments is limited by the ability to acquire information on regions containing these six residues.

Additives have been introduced into the reaction solution during footprinting to modify amino-acid side chains with other chemical moieties, such as glycine ethyl ester (GEE) for carboxylic acids (Gau et al., 2011), dimethyl (2-hydroxy-5-nitrobenzyl) sulfonium bromide (HNSB) for tryptophan (Borotto et al., 2017) and diethyl-pyrocarbonate. (Limpikirati et al., 2019). However, only a moderate increase in residue-level labeling coverage has been achieved. Recently, protein footprinting using trifluoromethyl radicals (•CF3) was developed (Cheng et al.). However, current protocols for footprinting using trifluoromethyl radicals generate •CF3 radicals using a laser or synchrotron beam and photolysis of water and/or hydrogen peroxide. To date no protocol exists for generating CF3 radicals for multiplexed labeling of proteins by plasma. The generation and utilization of multiple radical species produced by a plasma for simultaneous, complementary labeling has also not been demonstrated. Moreover, extensive labeling of less reactive residues remains challenging and can have negative effects on sequence coverage for applications such as epitope mapping. The methods described herein address these and other issues and provide a method for PLIMB-mediated protein footprinting with simultaneous labeling of multiple complementary chemical moieties, including hydroxyl (•OH), Nitro (•NO2) and trifluoromethyl (•CF3) radicals generated by a plasma to maximize labeling coverage and increase the resolution of protein footprinting data. It is demonstrated herein that CF3 radicals generated by plasma can label all 20 amino acids, thus significantly improving labelling coverage and increasing the resolution of protein footprinting data.

The number of radicals generated in a given footprinting experiment is a direct function of reaction conditions such as concentration of reactants, dose of the initiator and buffer composition. The source of radical initiation—laser photolysis, synchrotron, or a plasma—is a determining factor in efficacy of radical generation and protein labeling, and the resulting radical generation and footprinting platforms need to be optimized accordingly to the system at hand. Moreover, the fundamental mechanism of each technique (laser photolysis, synchrotron, or plasma) appears to generate different reaction conditions that are not necessarily translatable from one system to another. For example, when synchrotron radiation and water are utilized for photolysis of water and subsequent radical generation, photochemistry can be said to apply. Plasma-mediated radical generation, such as in PLIMB, involves a unique reaction environment, where the sample is subjected to both high-energy electrons under an electric potential and photons with energies that span a wide range of the electromagnetic spectrum. Therefore, both electrochemical and photochemical mechanisms of radical initiation can be said to apply. The resulting radical species generated by a plasma are therefore unique, and the chemical conditions for radical generation should be optimized accordingly for downstream footprinting experiments. Moreover, the resulting labeling should present a balance between maximizing the number of individual amino-acid residues labeled and population-level percent modification (i.e., average number of labels per protein molecule in a sample solution). If percent modification is too high, the protein may partially or fully denature during radical treatment, which will affect downstream data analysis and accuracy of structural characterization with the footprinting technique. The dynamic nature of multiple radicals being generated simultaneously also means that the platform should be assessed under the influence of radical scavengers, such that the system is robust and resilient against a plethora of multiple reaction conditions. Examples include but are not limited to the utilization of different buffers, pH, the presence or absence of drugs, radical scavengers, different concentrations of additives, and protein concentrations. Described herein is an optimized platform that addresses these and other concerns. In some aspects, provided herein is a method for hydroxyl (•OH), Nitro (•NO2) and trifluoromethyl (•CF3) radical generation by a plasma and multiplexed protein labeling using the same. The methods provided herein are optimized for variables such as the influence of reactant concentrations, buffer composition, and electrode geometry.

In some aspects, provided herein is a method of labeling a biological molecule with trifluormethyl radicals. In some embodiments, provided herein is a method of labeling a biological molecule with trifluoromethyl radicals comprising providing a sample comprising a trifluoromethyl radical precursor and a biological molecule, and generating a plasma in the sample, thereby converting the radical precursor into trifluoromethyl radicals which interact with and label the biological molecule. In some embodiments, the trifluoromethyl radical precursor is sodium triflinate. Accordingly, in some embodiments provided herein is a method of labeling a biological molecule with trifluoromethyl radicals, comprising providing a sample comprising sodium triflinate and a biological molecule and generating a plasma in a sample, thereby converting the sodium triflinate into trifluoromethyl radicals which interact with the biological molecule, thereby labeling the biological molecule. In some embodiments, the sample comprises 1 nM to 4 M sodium triflinate. In some embodiments, the sample comprises 50 uM to 500 mM sodium triflinate. In some embodiments, the sample comprises 1 mM to 100 mM sodium triflinate. In some embodiments, the sample comprises 10 mM to 50 mM sodium triflinate. In some embodiments, the sample comprises 1 mM to 100 mM sodium triflinate, 5 mM to 90 mM sodium triflinate, 10 mM to 80 mM sodium triflinate, 15 mM to 75 mM sodium triflinate, 20 mM to 70 mM sodium triflinate, 25 mM to 65 mM sodium triflinate, 30 mM to 60 mM sodium triflinate, 35 mM to 55 mM sodium triflinate, or 40 to 50 mM sodium triflinate.

In some aspects, provided herein is a method for multiplexed labeling of a biological molecule. In some embodiments, provided herein is a method for multiplexed labeling of a biological molecule with trifluoromethyl and hydroxyl and/or nitro radicals. In some embodiments, the method is performed in a single reaction vial. In some embodiments, labeling with the radicals is performed simultaneously. In some embodiments, the method comprises providing a sample comprising two or more radical precursors and a biological molecule, and generating a plasma in the sample. In some embodiments, generation of plasma in the sample converts the two or more radical precursors in the sample into two or more of trifluoromethyl radicals, hydroxyl radicals, and nitro radicals, which interact with the biological molecule and label the biological molecule. The process of radicals interacting with the biological molecule and labeling the biological molecule can also be referred to herein as “footprinting”, or “radical footprinting”.

In some embodiments, labeling with the radicals is performed sequentially. In some embodiments, the biological molecule is labeled with a first radical, and subsequently labeled with a second radical. In some embodiments, the biological sample is labeled with three or more radicals sequentially. In some embodiments, the method for multiplexed labeling of a biological molecule comprises providing a sample comprising a first radical precursor and a biological molecule and generating a plasma in the sample, thereby converting the first radical precursor in the sample into a first radical which interacts with the biological molecule, thereby labeling the biological molecule with the first radical. In some embodiments, the method further comprises adding a second radical precursor to the biological sample and generating a plasma in the sample, thereby converting the second radical precursor in the sample into a second radical which interacts with the biological molecule, thereby labeling the biological molecule with the second radical. In some embodiments, the method comprises providing a sample comprising a first radical precursor, a second radical precursor, and a biological molecule, and generating a first and second plasma in the sample. In some embodiments, the method comprises generating a first plasma in the sample, thereby converting the first radical precursor in the sample into a first radical which interacts with the biological molecule, thereby labeling the biological molecule with the first radical. In some embodiments, the method further comprises generating a second plasma in the sample, thereby converting the second radical precursor in the sample into a second radical which interacts with the biological molecule, thereby labeling the biological molecule with the second radical. In some embodiments, the first plasma and the second plasma are generated using different parameters (e.g. different voltages, different plasma doses, etc.). In some embodiments, the first radical precursor is sodium triflinate and the second radical precursor is hydrogen peroxide or tert-butyl hydroperoxide (TBHP). In some embodiments, the first radical precursor is hydrogen peroxide or tert-butyl hydroperoxide (TBHP) and the second radical precursor is sodium triflinate.

In some embodiments, the radical precursors comprise sodium triflinate and hydrogen peroxide (H₂O₂). In some embodiments, such as for simultaneous multiplexed labeling, the sodium triflinate and hydrogen peroxide are present in the sample and a plasma is generated in the sample to produce the radicals simultaneously. In some embodiments, such as for sequential multiplexed labeling, the first radical precursor (e.g. sodium triflinate) is present in the sample and a first plasma is generated, and following a suitable duration of time the second radical precursor (e.g. hydrogen peroxide) is added to the sample, and a second plasma is generated. Suitable concentrations are described below. Although the concentrations are described as being in the sample, it is understood that the concentrations below also appropriate for the amount of the first radical precursor present in the sample and the amount of the second radical precursor that is added to the sample. In some embodiments, the sample comprises 1 nM to 4 M sodium triflinate and 1 nM to 10 M hydrogen peroxide. In some embodiments, the sample comprises 50 uM to 500 mM sodium triflinate and 0.1 mM to 100 mM hydrogen peroxide. In some embodiments, the sample comprises 1 mM to 100 mM sodium triflinate and 1 mM to 100 mM hydrogen peroxide. In some embodiments, the sample comprises 10 mM to 50 mM sodium triflinate and 10 mM to 100 mM hydrogen peroxide. In some embodiments, the sample comprises 1 mM to 100 mM sodium triflinate, 5 mM to 90 mM sodium triflinate, 10 mM to 80 mM sodium triflinate, 15 mM to 75 mM sodium triflinate, 20 mM to 70 mM sodium triflinate, 25 mM to 65 mM sodium triflinate, 30 mM to 60 mM sodium triflinate, 35 mM to 55 mM sodium triflinate, or 40 to 50 mM sodium triflinate and 1 mM to 100 mM hydrogen peroxide, 2 mM to 90 mM hydrogen peroxide, 3 mM to 80 mM hydrogen peroxide, 4 mM to 70 mM hydrogen peroxide, 5 mM to 60 mM hydrogen peroxide, 6 mM to 50 mM hydrogen peroxide, 7 mM to 40 mM hydrogen peroxide, 7.5 mM to 35 mM hydrogen peroxide, 8 mM to 30 mM hydrogen peroxide, 8.5 mM to 20 mM hydrogen peroxide, or 10 mM to 20 mM hydrogen peroxide. In some embodiments, the sample comprises 10 mM to 50 mM sodium triflinate and 10 mM to 50 mM hydrogen peroxide. For example, in some embodiments the sample comprises 10 mM sodium triflinate, 15 mM sodium triflinate, 20 mM sodium triflinate, 25 mM sodium triflinate, 30 mM sodium triflinate, 35 mM sodium triflinate, 40 mM sodium triflinate, 45 mM sodium triflinate, or 50 mM sodium triflinate and 10 mM hydrogen peroxide, 20 mM hydrogen peroxide, 30 mM hydrogen peroxide, 40 mM hydrogen peroxide, or 50 mM hydrogen peroxide. In some embodiments, the sample comprises 50 mM sodium triflinate and 10 mM hydrogen peroxide.

In some embodiments, the two or more radical precursors comprise sodium triflinate and tert-butyl hydroperoxide (TBHP). In some embodiments, such as for simultaneous multiplexed labeling, the sodium triflinate and TBHP are present in the sample and a plasma is generated in the sample to produce the radicals simultaneously. In some embodiments, such as for sequential multiplexed labeling, the first radical precursor (e.g. sodium triflinate) is present in the sample and a first plasma is generated, and following a suitable duration of time the second radical precursor (e.g. TBHP) is added to the sample, and a second plasma is generated. Suitable concentrations are described below. Although the concentrations are described as being in the sample, it is understood that the concentrations below also appropriate for the amount of the first radical precursor present in the sample and the amount of the second radical precursor that is added to the sample. In some embodiments, the sample comprises 1 nM to 4 M sodium triflinate and 1 nM to 7.5 M TBHP. In some embodiments, the sample comprises 10 mM to 50 mM sodium triflinate and 0.1 mM to 100 mM TBHP. In some embodiments, the sample comprises 1 mM to 100 mM sodium triflinate and 50 μM to 50 mM TBHP. For example, in some embodiments the sample comprises 1 mM to 100 mM sodium triflinate, 5 mM to 90 mM sodium triflinate, 10 mM to 80 mM sodium triflinate, 15 mM to 75 mM sodium triflinate, 20 mM to 70 mM sodium triflinate, 25 mM to 65 mM sodium triflinate, 30 mM to 60 mM sodium triflinate, 35 mM to 55 mM sodium triflinate, or 40 to 50 mM sodium triflinate and 50 μM to 50 mM TBHP, 100 μM to 40 mM TBPH, 200 μM to 30 mM TBHP, 300 μM to 20 mM TBPH, 400 μM to 10 mM TBHP, 500 μM to 5 mM TBPH, 600 μM to 4 mM TBPH, 700 μM to 3 mM TBPH, 800 μM to 2 mM TBPH, or 900 μM to 1 mM TBPH. In some embodiments, the sample comprises 10 mM to 50 mM sodium triflinate and 100 μM to 10 mM TBHP. For example, in some embodiments the sample comprises 10 mM sodium triflinate, 15 mM sodium triflinate, 20 mM sodium triflinate, 25 mM sodium triflinate, 30 mM sodium triflinate, 35 mM sodium triflinate, 40 mM sodium triflinate, 45 mM sodium triflinate, or 50 mM sodium triflinate and 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM or 10 mM TBHP. In some embodiments, the sample comprises 50 mM sodium triflinate and 1 mM THBP.

In some embodiments, the sample additionally comprises a buffer. Exemplary buffers include, for example, phosphate buffered saline solution, tris(hydroxymethyl)aminomethane (tris), tris hydrochloric acid, ammonium bicarbonate, 4-(2-hydroxyethyl)-1-piperazineethanesulfonic acid (HEPES), 3-(N-morpholino)propanesulfonic acid (MOPS), 2-(N-morpholino)ethanesulfinic acid (MES), 2,2-Bis(hydroxymethyl)-2,2′,2″-nitrilotriethanol (bis-tris), N-(2-Acetamido)iminodiacetic acid (ADA), piperazine-N,N′-bis(2-ethanesulfonic acid) (PIPES). N-(2-Acetamido)-2-aminoethanesulfonic acid (ACES), 3-(N-morpholinyl)-2-hydroxypropanesulfonic acid sodium salt (MOPSO), 1,3-bis(tris(hydroxymethyl)methylamino)propane (bis-tris propane), N,N-Bis(2-hydroxyethyl)-2-aminoethanesulfonic acid (BES), 2-[[1,3-dihydroxy-2-(hydroxymethyl)propan-2-yl] amino] ethanesulfonic acid (TES), 3-(Bis(2-hydroxyethyl)amino)-2-hydroxypropane-1-sulfonic acid (DIPSO), 3-[[1,3-dihydroxy-2-(hydroxymethyl)propan-2-yl]amino]-2-hydroxypropane-1-sulfonic acid (TAPSO), Trizma, piperazine-1,4-bis(2-hydroxypropanesulfonic acid) dihydrate (POPSO), 3-[4-(2-Hydroxyethyl)-1-piperazinyl]propanesulfonic acid (HEPPS), N-(2-Hydroxy-1,1-bis(hydroxymethyl) ethyl) glycine (TRICINE), glycylglycine (GLY-GLY), 2-(Bis(2-hydroxyethyl) amino) acetic acid (BICINE). N-(2-hydroxyethyl)piperazine-N′-(4-butanesulfonic acid) (HEPBS), 3-[[1,3-dihydroxy-2-(hydroxymethyl)propan-2-yl]amino]propane-1-sulfonic acid (TAPS), 2-amino-2-methyl-1,3-propanediol (AMPD), N-(1,1-Dimethyl-2-hydroxyethyl)-3-amino-2-hydroxypropanesulfonic acid (AMPSO), N-cyclohexyl-2-aminoethanesulfonic acid (CHES), N-cyclohexyl-2-hydroxyl-3-aminopropanesulfonic acid (CAPSO), 1-amino-2-methyl-1-propanol (AMP), N-cyclohexyl-3-aminopropanesulfonic acid (CAPS), 4-(cyclohexylamino)-1-butanesulfonic acid (CABS). Lysogeny broth (LB) or other nutrient growth media, anything defined as a ‘biological buffer’, a biologically or physiologically-relevant salt, combinations thereof, and the like. In certain aspects, the buffer solution can have a pH value of between 1 and 14, including but not limited to, a pH of between 3 and 9, or a pH of between 4 and 8.

In some embodiments, the sample additionally comprises a buffer selected from phosphate buffered saline (PBS) or HEPES buffer. In some embodiments, the sample comprises PBS. In some embodiments, the sample comprises 50-100 mM PBS. For example, in some embodiments the sample comprises 50 mM PBS, 60 mM PBS, 70 mM PBS, 80 mM PBS, 90 mM PBS, or 100 mM PBS. In some embodiments, the sample comprises PBS having a pH of 7.4 In some embodiments, the sample comprises HEPES buffer. In some embodiments, the sample comprises 5-50 mM HEPES buffer. For example, in some embodiments the sample comprises 5 mM HEPES, 10 mM HEPES, 15 mM HEPES, 20 mM HEPES, 25 mM HEPES, 30 mM HEPES, 35 mM HEPES, 40 mM HEPES, 45 mM HEPES, or 50 mM HEPES. In some embodiments, the HEPES buffer has a pH of 6.0. In some embodiments, the HEPES buffer has a pH of 7.4.

Exemplary systems and methods for generating plasma are described in PCT Patent Publication No. WO2017201457A1, the entire content of which is incorporated herein by reference for all purposes. In some embodiments, plasma is generated by a plasma electrode. In some embodiments, the plasma is generated by a plasma electrode comprising a plasma source point. The “plasma source point” refers to the point on the electrode from which the plasma emerges. In some embodiments, the plasma electrode comprises a dielectric coating that prevents direct contact between the plasma electrode and the sample. In some embodiments, the dielectric coating covers at least the plasma source point. In some embodiments, the plasma electrode is a component of a plasma jet, which refers to a device which generates a plasma within a first space and propels the generated plasma towards a target (e.g. the sample) by movement of gas through the jet and the shaping of the plasma jet.

In some embodiments, plasma is generated by a system including a ground electrode, a plasma electrode, and a power supply. In some embodiments, electrical power from the power supply is used to generate a high voltage signal, whether alone or in combination with an amplifier. In some embodiments, plasma is formed when the voltage between the plasma electrode and the ground electrode exceeds a given value. The value may be dependent on the voltage magnitude, the frequency of the signal, and/or the space between the plasma electrode and the ground electrode.

In some embodiments, plasma can be generated by a voltage of between 1 volt (V) and 1 megavolt (MV). For example, in some embodiments plasma is generated by a voltage between 1V and 1 MV, between 100V and 750 kilovolts (kV), between 500 V and 500 kV, between 1 kV and 100 kV, between 1 kV and 50 kV, or between 5 kV and 15 kV.

In some embodiments, the plasma source point of the plasma electrode is placed within the sample. In some embodiments, the plasma is generated from a plasma source point placed within the sample. In some embodiments, the plasma source point of the plasma electrode is placed at the interface of a gas and the sample. In some embodiments, plasma is generated from a plasma source point placed at an interface of a gas and the sample, and the plasma then travels from the gas to the sample. In some embodiments, the gas is air. In some embodiments, the gas (e.g. air) refers to gas comprising both oxygen and nitrogen. In some embodiments, the sample is in contact with a fluid, and plasma is generated at the interface of fluid and the sample. In some embodiments, the sample is in contact with a fluid, and plasma is generated at the interface of gas (e.g. air) and the fluid, which plasma then passes through the fluid and into the sample. “Generating a plasma in a sample” is inclusive of any of the above-described mechanisms. In some embodiments, the plasma source point is placed (and thus plasma is generated) within 10 cm of the sample. In some embodiments, the plasma source point is placed within 3 cm of the sample. The plasma will be generated in the gas (e.g. air) and pass onto the sample. For example, in some embodiments the plasma electrode is placed at the interface of gas and the sample, within 3 cm, within 2.5 cm, within 2 cm, within 1.5 cm, within 1 cm, or within 0.5 cm of the sample.

The plasma can be generated for any suitable duration of time. In some embodiments, the plasma is generated for generated for 1 ns to 72 hours. For example, in some embodiments the plasma is generated for 1 ns to 72 hours, 100 ns to 24 hours, 1500 ns to 12 hours, 1 second to 6 hours, 1.5 seconds to 1 hour, 2 seconds to 30 minutes, 3 seconds to 10 minutes, 4 seconds to 5 minutes, or 5 seconds to 2 minutes. In some embodiments, the plasma is generated for 5 seconds to 120 seconds. For example, in some embodiments the plasma is generated for 5 seconds to 120 seconds, 10 seconds to 100 seconds, 20 seconds to 90 seconds, 30 seconds to 80 seconds, 25 seconds to 75 seconds, 30 seconds to 70 seconds, 40 seconds to 60 seconds, or about 50 seconds. The duration of plasma generation can be controlled by the power supply, which can turn off power to the plasma electrode after the desired duration of plasma generation has occurred.

In some embodiments, generation of plasma in the sample converts the two or more radical precursors in the sample into two or more of trifluoromethyl radicals, hydroxyl radicals, and nitro radicals, which interact with the biological molecule and label the biological molecule. In some embodiments, generation of plasma in the sample converts the two or more radical precursors (e.g. sodium triflinate and hydrogen peroxide, or sodium triflinate and TBHP), into trifluoromethyl radicals (CF3), hydroxyl radicals (OH), and nitro radicals (NO2). In some embodiments, the trifluoromethyl radicals, hydroxyl radicals, and nitro radicals modify (label) the biological molecule, a process referred to as “radical footprinting”. In some embodiments, generation of plasma in the sample converts the first radical precursors in the sample into a first radical which interact with the biological molecule and label the biological molecule. In some embodiments, generation of a second plasma in the sample after addition of a second radical precursor converts the second radical precursor into a second radical, which interacts with and labels the biological molecule. In some embodiments, the biological molecule is already labeled with the first radical. In some embodiments, the trifluoromethyl radicals, hydroxyl radicals, and nitro radicals modify (label) the biological molecule, a process referred to as “radical footprinting”.

In some embodiments, the method further comprises quenching the reaction (e.g. after generating the plasma in the sample). For example, in some embodiments the method further comprises quenching the sample with a solution comprising a radical quencher. In some embodiments, the radical quencher is a sulfur-containing amino acid, such as methionine, cystine, homocysteine, or taurine. In some embodiments, the method further comprises quenching the sample with a solution comprising 10 mM to 500 mM methionine. For example, in some embodiments the method comprises quenching the sample with a solution comprising 10 mM to 500 mM methionine, 25 mM to 450 mM methionine, 50 mM to 400 mM methionine, or 75 mM to 375 mM methionine, 100 mM to 350 mM methionine, 150 mM to 300 mM methionine, or about 250 mM methionine.

In some embodiments, the sample comprises one or more radical scavengers (e.g. radical scavengers are present in the sample before and/or during generation of plasma). Radical scavengers refer to any suitable substance that removes or deactivates free radicals in the sample. Suitable radical scavengers include, for example, methionine, cystine, homocysteine, taurine, DMSO, catalase, glucose, surfactants, buffers, histidine, DTT, TCEP, lipids, sucrose, etc. In some embodiments, the sample comprises DMSO. In some embodiments, the sample comprises 2% or less DMSO.

In some aspects, the biological molecule is a nucleic acid molecule, a protein, a lipid, or a biological metabolite. In some embodiments, the biological molecule comprises a protein. In some embodiments, the sample comprises more than one biological molecule. For example, in some embodiments the sample comprises two or more different proteins of interest. In some embodiments, the protein has been digested using one or more cleavage factors. In some embodiments, the method further comprises identifying labeling of the biological molecule (e.g. evaluating multiplexed labeling of the biological molecule with the trifluoromethyl radicals, hydroxyl radicals, and nitro radicals) using mass spectrometry.

The methods described herein can be utilized to determine structural information about a biological molecule. Biological molecules can include secondary, tertiary, and quaternary structure that precludes solvent interaction with various parts of the biological molecule. In some embodiments, the methods described herein can be used to determine whether a portion of a biological molecule is accessible to a solvent. Labeling with a radical as described herein indicates that the portion of the biological molecule is accessible, whereas the absence of labeling indicates that the portion is inaccessible. In some embodiments, the methods can be used to assess a biological molecule having solvent accessible positions and solve inaccessible positions. For example, a cleavage factor can be introduced to introduce a predictable change in structure of the biological molecules, and a comparison of radical labeling in the protein after contact with the cleavage factor can be compared to radical labeling in the protein or in an equivalent protein not contacted with the cleavage factor to provide information about accessibility.

Exemplary cleavage factors include proteases and peptidases. Nonlimiting examples of suitable cleavage factors include Trypsin (e.g., bovine). Chymotrypsin (e.g., bovine), Endoproteinase Asp-N (e.g., Pseudomonas fragi), Endoproteinase Arg-C (e.g., mouse submaxillary gland), Endoproteinase Glu-C (e.g., V8 protease) (e.g., Staphylococcus aureus), Endoproteinase Lys-C (e.g., Lysobacter enzymogenes), Pepsin (e.g., porcine), Thermolysin (e.g., Bacillus thermo-proteolyticus), Elastase (e.g., porcine), Papain (e.g., Carica papaya), Proteinase K (e.g., Tritirachium album), Subtilisin (e.g., Bacillus subtilis), Clostripain (endoproteinase-Arg-C) (e.g., Clostridium histolyticum), Exopeptidase, Carboxypeptidase A (e.g., bovine), Carboxypeptidase B (e.g., porcine), Carboxypeptidase P (e.g., Penicillium janthinellum), Carboxypeptidase Y (e.g., yeast), Cathepsin C, Acylamino-acid-releasing enzyme (e.g., porcine), Pyroglutamate aminopeptidase (e.g., bovine), and the like.

The methods described herein can be useful for a variety of applications, including quality control for biopharmaceuticals, such as determining whether a biopharmaceutical has retained its secondary, tertiary, and/or quaternary structure. The methods described herein can be used in methods of assessing a disease state in a subject, such as diseases caused by a conformational change in one or more biological molecules in a subject. For example, if a disease state is expressed by the breaking apart of a protein dimer, the methods of the present disclosure could be used to identify that the contact surfaces between the subunits of the dimer, which are normally not accessible to solvent, have become accessible to solvent. The methods described herein can also be utilized to study temperature-dependent properties of a sample of interest. For example, kinetics, protein folding, and other temperature-dependent mechanisms of interest can be studied with temperature-dependent deployment of the methods described herein. The methods described herein can be utilized to determine a rate of modification for components or sub-components within the sample of interest. For example, the methods described herein can compare the rate of oxidation (e.g. radical labeling) of two different residues on a protein of interest and can make various subsequent deductions based on the differences between those rates, such as determining a level of solvent accessibility.

In some aspects, provided herein are kits for use in the methods described herein. For example, in some aspects provided herein are kids comprising two or more radical precursors and a buffer. Suitable radical precursors and buffers are described above. For example, in some embodiments the two or more radical precursors are sodium triflinate and hydrogen peroxide and the buffer comprises PBS or HEPES buffer. As another example, in some embodiments the two or more radical precursors comprise sodium triflinate and tert-butyl hydroperoxide (TBHP) and the buffer comprises PBS or HEPES buffer.

### EXAMPLES

### Example 1

### Simultaneous, Multiplexed PLIMB-Mediated Hydroxyl (•OH), Nitro (•NO2) and Trifluoromethyl (•CF3) Labeling for Protein-Footprinting in a Single Reaction Vial

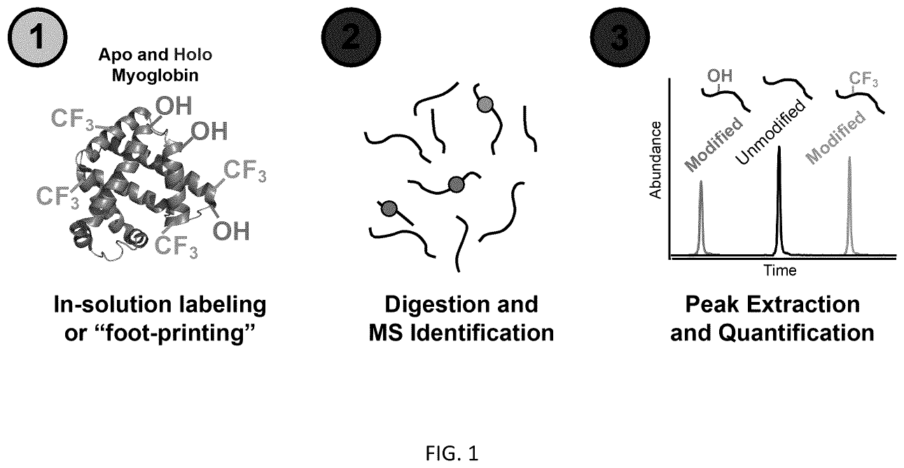

Demonstrated herein are methods for PLIMB-mediated simultaneous hydroxyl (•OH), Nitro (•NO2) and trifluoromethyl (•CF3) labeling of proteins in solution in a single reaction vial. The experiments described herein demonstrate the development of a method for multiplexed radical footprinting strategy to improve the coverage and resolution of protein footprinting and MS-structural studies. The multiplexed radical footprinting platform was shown to be effective at labeling holo and apo myoglobin. An schematic showing an overview of exemplary steps is shown in FIG. 1. The method described herein improves the resolution of •OH footprinting.

### Methods

Materials and Reagents: All chemicals and reagents were purchased from Sigma-Aldrich unless otherwise noted. Trypsin, Trypsin/LysC, Glu-C and PNGaseF and were purchased from Promega. Optima LC-MS grade acetonitrile, water and 0.1% formic acid in water were purchased from Fisher Scientific. pH paper was purchased from Fisher Scientific.

Experimental sample preparation conditions: Horse, skeletal myoglobin (apo and holo) was resuspended in 5 mM PBS. pH 7.4 in a total volume of 50 uL. Various concentrations of sodium triflinate (0 mM, 5 mM, 200 mM, 1M) and hydrogen peroxide (0 mM, 1 mM, 100 mM) were utilized. Replicates were treated with PLIMB for 0, 5, 10 or 30 seconds. Following exposure, 25 mM methionine was added to quench the reaction. Samples were precipitated with TCA, followed by acetone. Dried proteins were then solubilized in 8M urea/50 mM ammonium bicarbonate (ABC), reduced with 5 mM DTT for 30 minutes at 50° C. and alkylated with 15 mM IAA in the dark at RT for 30 minutes. Prior to digestion samples were diluted to 1 M urea with 50 mM ABC and trypsin/LysC was added to a ratio of 1:25 (protein: enzyme) overnight at 37° C.

Experimental LC-MS/MS conditions: Dried-down peptides were resuspended into 0.1% formic acid injected into the LC/MS system for spectral analysis. The samples were analyzed using data-dependent acquisition with an Vanquish Neo coupled to an Orbitrap Elite 240 mass spectrometer (Thermo Scientific). A 30-minute chromatographic gradient from 2 to 40% acetonitrile with 0.1% formic acid was used for separation over a 2 μM, 15 cm Easy-Spray PepMap C18 column (ThermoFisher Scientific). A top-20 data-dependent acquisition was performed MS1 parameters of 120K resolving power in the Orbitrap, a scan range of 375-1200 m/z, a normalized AGC target of 300%, and MS² parameters of charge state 2-6 selection, a quadrupole isolation window of 2 Da, HCD collision energy of 30%, a normalized AGC value of 50%, and an automatic scan range starting at 110 m/z. Dynamic exclusion of 20 seconds was used after seeing an ion once. In cases where more coverage was needed a targeted or data-independent acquisition approach was utilized.

Data Analysis for PLIMB-mediated OH, CF3 and NO2 protein footprinting: The ‘.raw’ MS and MS/MS data files were searched against the protein fasta sequence using Protein Metrics Byos™ for peptide spectral matching and label-free quantification, with a 1% false discovery rate (FDR) cutoff. A list of baseline expected modifications and expected PLIMB modifications was utilized in the database search. The following modifications were used in the search. Baseline modifications: Carbamidomethyl/+57.021464 Da @ C (fixed). PLIMB-mediated variable modifications: Oxidation/+15.994915 Da @ C, F, H, M, W, Y, Dioxidation/+31.989829 Da @ C, F, M, W, Y, Cys-Oxidation/+15.994915-57.021464 Da @ C, Cys-Dioxidation/+31.989829-57.021464 Da @ C, Cys-Trioxidation/+47.984745-57.021464 Da @C, Nitro/+44.985078 Da @ W, Y, Trifluoromethylation/+67.9874 Da @ A, C, D, E, F, G, H, I, K, L, M, N, P, Q, R, S, T, V, W, Y. The proportion of plasma-modified peptide (marked as expected variable PLIMB-modifications above) was calculated based on extracted ion chromatogram (XIC) relative peak areas of modified versus total peptide signal, including modified and unmodified peptides. Residue level quantification was utilized when chromatographic separation and sufficient flanking MS/MS fragments for the amino acid were present. All identifications and integrated areas were manually validated.

### Results

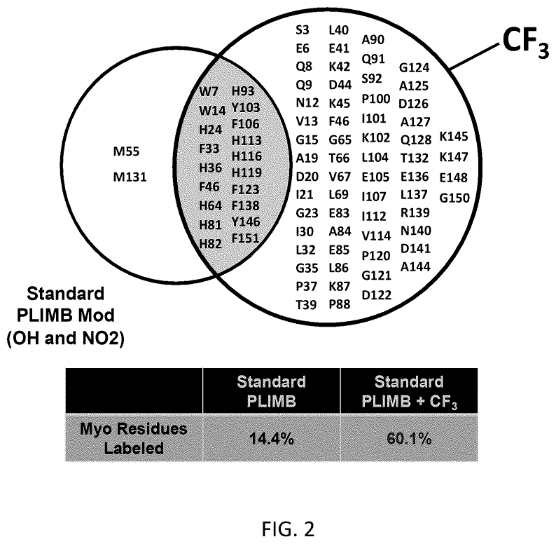

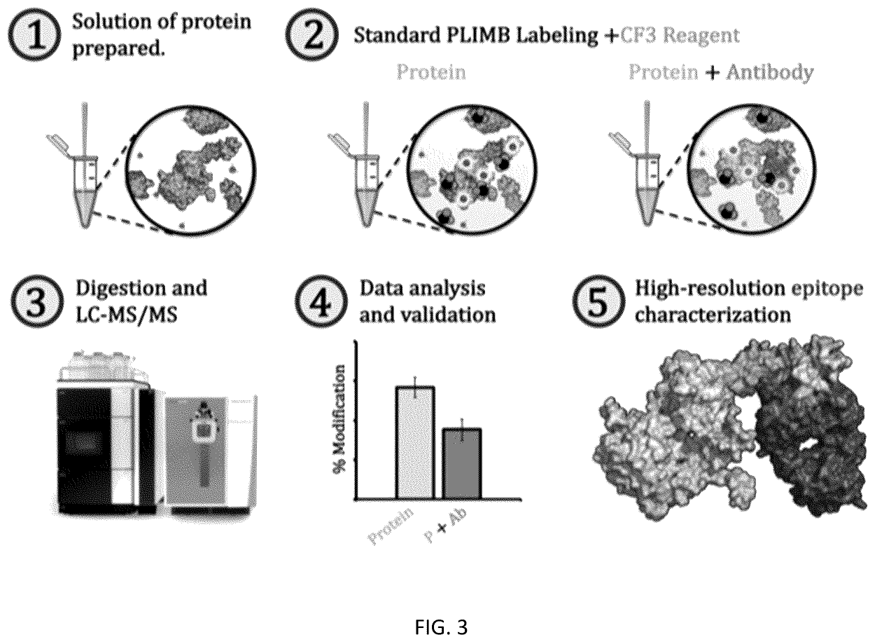

First, it was demonstrated that PLIMB treatment in the presence of the sodium triflinate reagent and hydrogen peroxide additives, produced hydroxyl (•OH), Nitro (•NO2) and trifluoromethyl (•CF3) radicals to effectively label myoglobin for downstream footprinting analysis. Next, the difference in labeling between PLIMB-mediated hydroxyl (•OH) radicals and trifluoromethyl (•CF3) radicals was compared (FIG. 2). Dual labeling of myoglobin using PLIMB treatment in solution with sodium triflinate provided an overall higher sequence coverage than hydroxyl radical labeling in water alone. Together, oxidative labeling of every amino acid was observed, including those normally difficult to selectively label such as lysine and glycine. While some overlap in labeling between residues existed (phenylalanine, tyrosine, and histidine), residues normally “silent” to •OH labeling (alanine, aspartic acid, glycine, valine, lysine, glutamic acid, glutamine, leucine, isoleucine, threonine, proline, serine, arginine, and asparagine) were all effectively labeled with •CF3 radicals. The only residue unique to •OH labeling was methionine. Overall, •CF3 and •OH labeling is highly complementary, as they exhibit different reactivities, resulting in a higher degree of amino acid labeling than was seen with either approach alone. Chromatographic separation between peptides with a single variable modification such as (OH), was utilized with MS/MS data to validate residue level labeling site positions. Targeted MS/MS was utilized in cases where assignment was initially ambiguous.

### Example 2

### PLIMB-Mediated Hydroxyl (•OH), Nitro (•NO2) and Trifluoromethyl (•CF3) Protein-Footprinting for HER2/Herceptin Epitope Mapping in a Single Reaction Vial

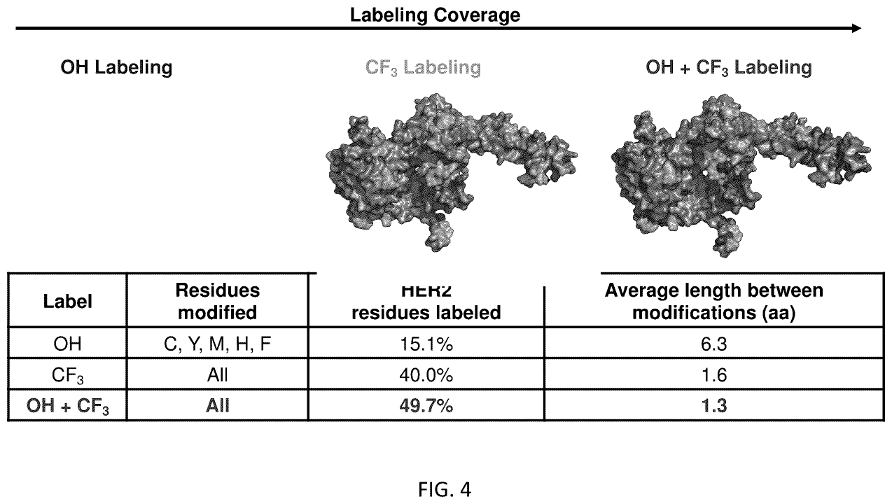

Mapping the binding region of an antigen to its corresponding antibody with high accuracy and resolution is of paramount importance to improve drug development success rates and decrease speed to market. To compare and test the resolution of (•OH) footprinting vs. the PLIMB-mediated multiplexed hydroxyl (•OH), Nitro (•NO2) and trifluoromethyl (•CF3) footprinting platform, human epidermal growth factor receptor-2 (HER2) was used alone or in complex with the therapeutic Herceptin Mab. A schematic showing exemplary methods for evaluating epitope mapping is shown in FIG. 4.

### Methods

Experimental conditions for sample preparation and PLIMB treatment: Prior to epitope mapping, the level of antigen and Mab protein modification in response to several PLIMB-induced hydroxyl radical exposure doses was monitored. Samples were prepared, and exposed to PLIMB for 0, 20, and 40 seconds. Subsequent PLIMB doses were chosen for optimal, balanced labeling of hydroxyl (•OH), Nitro (•NO2) and trifluoromethyl (•CF3).

Experimental Conditions for sample digestion and clean-up: Solutions of HER2 alone and HER2/Herceptin added at a 1:1 molar ratio were solubilized in 5 mM PBS, pH 7.4 in addition to 5 mM PBS, pH 7.4, 10 mM H2O2 and 50 mM sodium triflinate and incubated at room temperature for 45 minutes. Solutions were aliquoted into 50 uL replicates for 20 and 40 seconds of PLIMB treatment. Following exposure, 25 mM methionine was added to quench the reaction. Samples were precipitated with TCA, followed by acetone. Dried proteins were then solubilized in 8M urea/50 mM ammonium bicarbonate (ABC), reduced with 5 mM DTT for 30 minutes at 50° C. and alkylated with 15 mM IAA in the dark at RT for 30 minutes. Prior to digestion samples were diluted to 1 M urea with 50 mM ABC and incubated overnight with PNGase F at 37° C. for deglycosylation. Trypsin/LysC was added to a ratio of 1:25 (protein: enzyme) for overnight digestion at 37° C. Chymotrypsin was added at a ratio of 1:25 (protein: enzyme) and proteins were digested further overnight at 37° C. Samples were then desalted and concentrated using C18 OMIX tips (Agilent).

Experimental conditions for LC-MS/MS: Dried-down peptides were resuspended into 0.1% formic acid at a concentration of 100 ng/μl and injected into the LC/MS system for spectral analysis. A 30-minute chromatographic gradient from 2 to 40% acetonitrile with 0.1% formic acid was used for separation over a 2 μM, 15 cm Easy-Spray PepMap C18 column (ThermoFisher Scientific). A top-20 data-dependent acquisition was performed MS1 parameters of 120K resolving power in the Orbitrap, a scan range of 375-1200 m/z, a normalized AGC target of 300%, and MS² parameters of charge state 2-6 selection, a quadrupole isolation window of 2 Da, HCD collision energy of 30%, a normalized AGC value of 50%, and an automatic scan range starting at 110 m/z. Dynamic exclusion of 20 seconds was used after seeing an ion once. In cases where more coverage was needed a targeted or data-independent acquisition approach was utilized.

Data analysis for HER2 Epitope Mapping: Differences in solvent accessibility of various regions of the antigen (HER2) using PLIMB and hydroxyl radical labeling were measured. First, two solutions were prepared to map the HER2 epitope: one with HER2 alone and the other with the Herceptin antibody added at a 1:1 antibody/antigen molar ratio. Samples of HER2 alone are referred to herein as “unbound,” and samples with antibody added are referred to herein as “bound” or “complex”. First, for epitope mapping, a peptide level-analysis was performed for detection of larger-order structural changes and identification of regions of interest showing “protection” and “deprotection” upon complexation. Peptides with a decreased level of modification upon binding with the Mab indicate “protection” or candidate epitope regions. Peptides with an increased level of modification upon complexation represent “deprotection” or areas of conformational change, which take place due to Mab binding. The peptides of interest were further validated, and residue-localized modifications were validated in Byos by verifying “Mod,” and “Delta-Mod” scores, visually inspecting MS² spectra, and adjusting extracted ion chromatogram (XIC) windows across peaks that showed consistency between samples and whose modifications could be identified with MS² hits. The residue-level analysis was utilized for peptides in the candidate epitope regions to determine which of the individually labeled amino acids show the greatest changes in solvent accessibility due to binding.

### Results

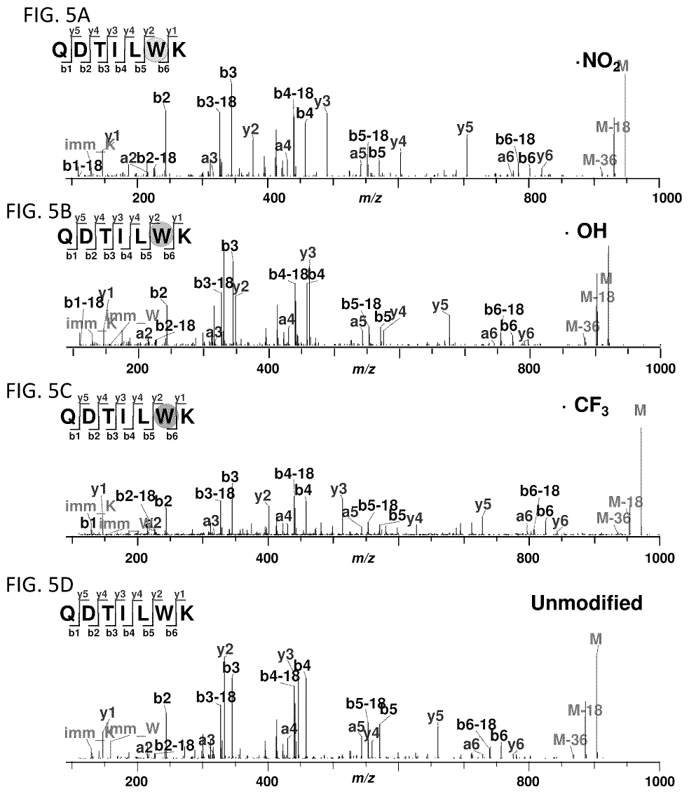

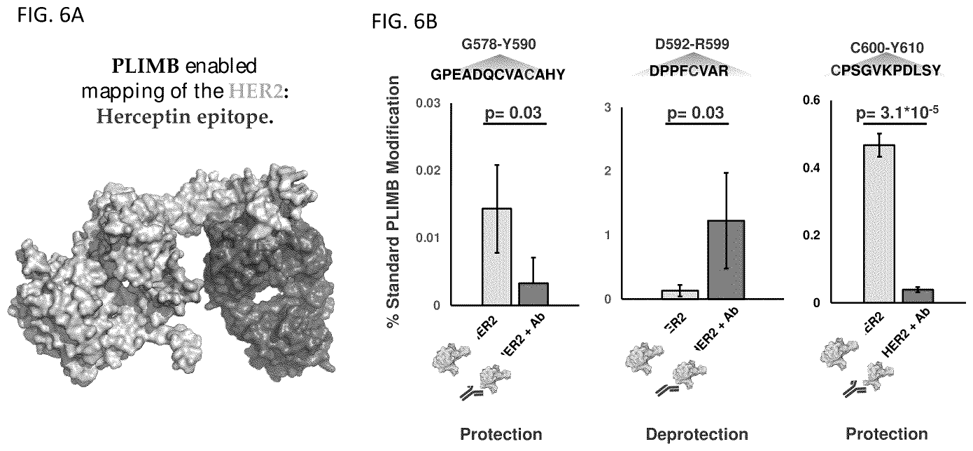

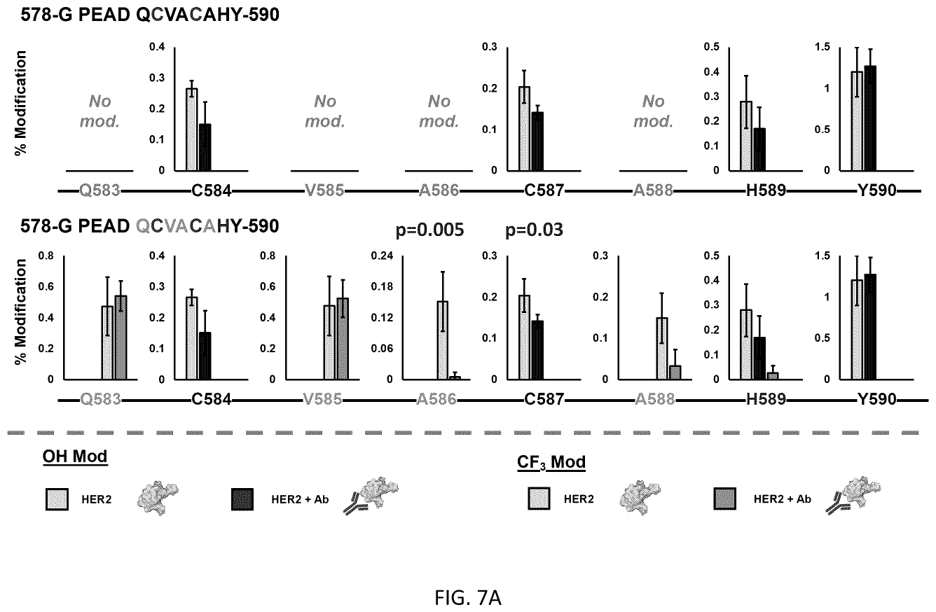

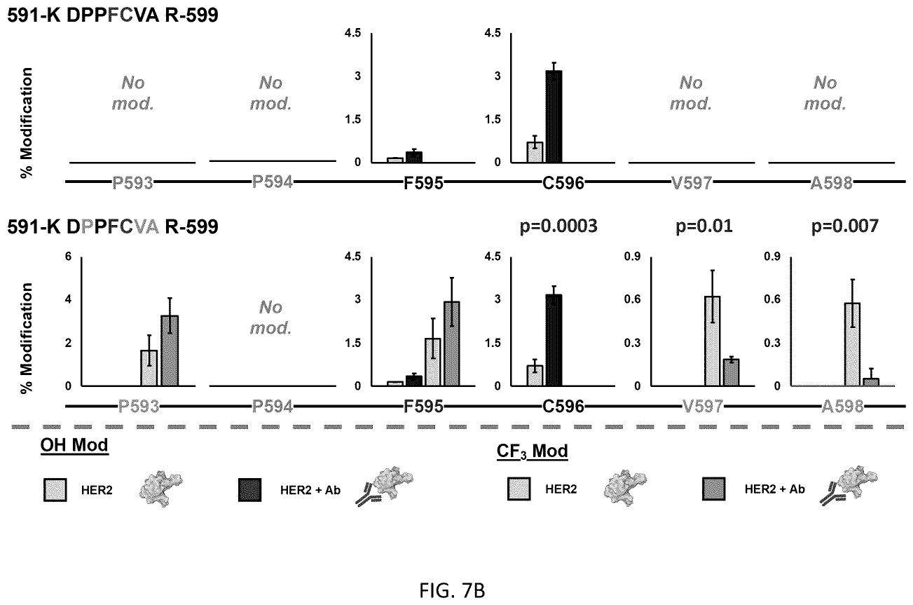

First, the residue level labeling coverage of the HER2 antigen between PLIMB-mediated hydroxyl (•OH) footprinting and the multiplexed labeling platform was compared (FIG. 4). Notably, while the hydroxyl radical footprinting condition labeled the expected cysteine, methionine, tyrosine, histidine, and phenylalanine residues, the (•CF3) radical labeled effectively labeled the remaining 15/20 amino acids (FIG. 4). Overall, the multiplexed labeling method provided an increase in labeling covering from 15.1% to 49.7% of the HER2 antigen residues (FIG. 4). Peptides with a single variable modification such as (OH). (NO2) or (CF3) were localized with MS/MS data to validate residue level labeling site positions and compare to the unmodified species (FIG. 5A-D). The multiplexed labeling allowed percent modification data to be collected with all three labels in the case of tryptophan (W), increasing the confidence of data and footprinting experiment (FIG. 5A-D). Next, a peptide-level analysis was performed across the entire length of the HER2 primary sequence for PLIMB treated samples prepared with and without the addition of the Herceptin Mab to detect differences in solvent accessibility by monitoring levels of modification upon binding/complexation (FIG. 6). Several HER2 peptides (G578-Y590, D592-R599 and C600-Y610) showed statistically significant differences in modification (protection) upon binding to the Herceptin Mab using the PLIMB-mediated hydroxyl (•OH) platform and were mapped to the cryo-EM structure (PDB:6OGE). The resulting epitope mapped with the PLIMB-mediated hydroxyl (•OH) platform overlaps and is consistent with the previously published structure, indicating a high level of accuracy. Next, to access the differences between the PLIMB-mediated OH radical vs. dual PLIMB-mediated multiplexed hydroxyl, nitro, and trifluoromethyl radical footprinting platform, a comparison of residue-level labeling was performed across the HER2 epitope regions (FIGS. 7A-7B). For the [G578-Y590] HER2 peptide, quantification of an additional five residues across the epitope region was obtained with trifluoromethyl radicals including residues normally “silent” to OH labeling such as alanine and valine (FIG. 7A). While cysteine and phenylamine labels were quantified using OH labeling for the HER2 peptide [K591-R599] spanning the second epitope region (2/6 residues of peptide), an additional three residues including proline, alanine and valine were quantified (5/6 residues of peptide) utilizing the multi-radical PLIMB-mediated labeling platform. In summary, the PLIMB-mediated multiplexed labeling platform both enhanced labelling coverage and significantly increased the resolution of footprinting for the application of epitope mapping.

### Example 3

### Sub-Residue Resolution PLIMB-Mediated Trifluoromethyl (•CF3) Protein Footprinting of TNFα

To further demonstrate the advantages of the PLIMB-mediated multiplexed hydroxyl (•OH), Nitro (•NO2) and trifluoromethyl (•CF3) footprinting platform, tumor necrosis factor alpha (TNFα) was treated to show sub-residue level labeling resolution and coverage obtained.

### Methods

Experimental Conditions for sample preparation, PLIMB treatment, digestion and clean-up: Solutions of TNFα (Acro Biosystems) were solubilized in 5 mM PBS, pH 7.4, 10 mM H2O2 and 50 mM sodium triflinate. Solutions were treated with 40 s of PLIMB. Following exposure, 25 mM methionine was added to quench the reaction. Samples were precipitated with TCA, followed by acetone. Dried proteins were then solubilized in 8M urea/50 mM ammonium bicarbonate (ABC), reduced with 5 mM DTT for 30 minutes at 50° C. and alkylated with 15 mM IAA in the dark at RT for 30 minutes. Prior to digestion samples were diluted to 1 M urea with 50 mM ABC and trypsin/LysC was added to a ratio of 1:25 (protein: enzyme) for overnight digestion at 37° C. Samples were diluted further to 0.18 M urea with 50 mM ABC and Glu-C was added at a ratio of 1:25 (protein: enzyme) and proteins were digested further overnight at 37° C. Samples were then desalted and concentrated using C18 OMIX tips (Agilent).

Experimental conditions for LC-MS/MS: Dried-down peptides were resuspended into 0.1% formic acid at a concentration of 100 ng/μl and injected into the LC/MS system for spectral analysis. A 30-minute chromatographic gradient from 2 to 40% acetonitrile with 0.1% formic acid was used for separation over a 2 μM, 15 cm Easy-Spray PepMap C18 column (ThermoFisher Scientific). A top-20 data-dependent acquisition was performed MS1 parameters of 120K resolving power in the Orbitrap, a scan range of 375-1200 m/z, a normalized AGC target of 300%, and MS² parameters of charge state 2-6 selection, a quadrupole isolation window of 2 Da, HCD collision energy of 30%, a normalized AGC value of 50%, and an automatic scan range starting at 110 m/z. Dynamic exclusion of 20 seconds was used after seeing an ion once. In cases where more coverage was needed a targeted or data-independent acquisition approach was utilized.

### Results

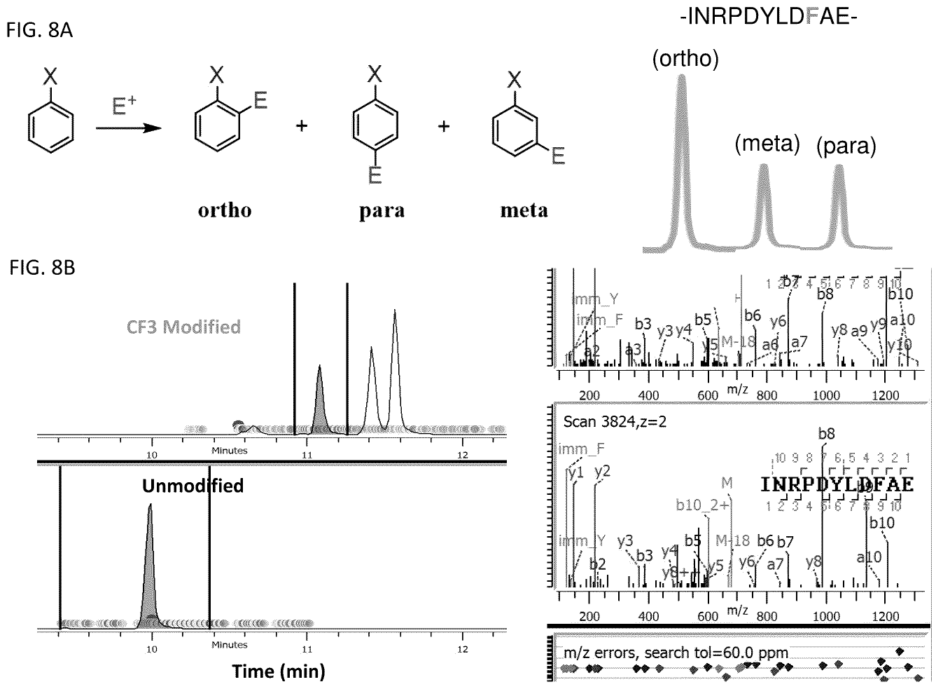

PLIMB-mediated CF3 radicals appeared to modify multiple positions on aromatic residues (such as phenylalanine) and generate different regioselective product chromatographic peaks (ortho, para, meta) for TNFα peptides (FIG. 8A). For example, utilizing targeted MS/MS, it was observed that for a single peptide containing a CF3 modified phenylalanine (-INRPDYLDFA-), three resultant product extracted ion chromatograms peaks were observed (FIG. 8B). Targeted MS/MS was used over the entire elution range to validate that each peak contained the same CF3 modified peptide, with varying levels of hydrophobicity (FIG. 8B). Given that radical substitution to a ring can result in different regioselectivity, this data strongly suggests that each peak represents an ortho, meta or para substituted product. Fractions can be collected to validate these products/the elution order using NMR. This work demonstrates that multiplexed labeling increases the resolution drastically, to a sub-residue level. The ability to finally tune resolution and detect changes in solvent accessibility of a drug-protein or protein-protein interactions to the sub-residue level may be highly advantageous for certain footprinting applications.

Optimization of platform conditions. Varying sample conditions of the PLIMB-mediated radical footprinting platform can tune the chemistry and reactivity of the resulting labeling. To assess the CF3 labeling of proteins, prevent unfolding, and further understand reaction conditions of PLIMB-mediated multi-radical labeling in solution, the following areas were identified and assessed for further experimentation and improvement: PLIMB dose, sodium triflinate (CF3 reagent) concentration, buffer composition, hydrogen peroxide concentration. Additionally, several new aspects from the optimized standard platform (10 mM hydrogen peroxide, 50 mM sodium triflinate in 5 mM PBS. pH 7.4) were investigated including, utilization and concentration of tert-butyl hydrogen peroxide (TBHP), electrode position (suspended vs. submerged), the presence/absence and concentration of radical scavengers and the influence of pH and the presence/absence of transition metal catalysts.

Optimization of PLIMB dose for PLIMB-mediated CF3 labeling. The optimization of PLIMB dose is essential for balancing the generation of the OH, NO2 and CF3 radicals in solution and monitoring the amino-acid residues they label. Previously, a linear dose response was observed for solvent exposed methionine on BSA treated with various PLIMB times. (Minkoff et al., 2017). This is expected, as an increased plasma dose is expected to introduce more hydroxyl radicals and hence more downstream labeling. However, in the multiplexed platform, given the plethora of radical species in solution, their interplay and dependance on one another, and their complicated generation mechanisms and varying half-lives, it is unlikely that a linear dose response of CF3 labeling versus the plasma dose would be observed. To further understand the effect of PLIMB dose on PLIMB-mediated OH, CF3 and NO2 radical generation, the baseline protein lysozyme was used with a fixed concentration of 1-1202 (10 mM) and sodium triflinate (50 mM) in solution. Optimization experiments led to the determination that varying PLIMB doses allowed for a different reaction environment (concentration of radical species present) and thus different relative amounts of the downstream labeling. Fundamentally, this data illustrates the complexity of the PLIMB-mediated radical reaction environment and an ability to tune and optimize the data depending on the downstream application and desired balance of modification. Optimization experiments led to the determination that 10-120 seconds of PLIMB exposure was sufficient for the multiplexed PLIMB-mediated footprinting platform for observation of all three labels (OH, CF3, and NO2), although 75 s yielding the highest level of all three modifications.

### Example 4

### PLIMB-Mediated CF3 Labeling of Lysozyme Treated with Variable Plasma Doses

Experimental Conditions for optimization of PLIMB dose for CF3 footprinting: Solutions of lysozyme (from chicken egg white) were prepared in PBS buffer, pH 7.4 with 10 mM H2O2 and 50 mM sodium triflinate in 50 uL replicate aliquots. Three replicates were exposed to varied PLIMB doses (10, 60, 75, 90, and 120 seconds). Following PLIMB exposure, samples were quenched with a 5 uL solution of 250 mM methionine in PBS, pH 7.4. Samples were then digested and analyzed by LC-MS/MS.

Results for optimization of PLIMB dose for PLIMB-mediated CF3 labeling: After mass spectrometry analysis, a peptide-level analysis of lysozyme was performed across reaction conditions, which allowed for quantification and characterization of percent PLIMB-mediated OH, CF3 and NO2 radical labeling as a function of PLIMB dose (10-120 s). The peptide-level analysis shows that after 10 seconds of PLIMB exposure, significant levels of every modification (OH, CF3 and NO2) are detected across every region of lysozyme, yielding a baseline for future experiments (FIG. 8A-8C). Additionally, a clear linear response in OH, NO2 or CF3 labeling with increasing PLIMB dose was not observed (FIG. 8A-C). Interestingly, certain doses lead to an increase or decrease in relative modification percent between the different labels (FIG. 8A-C).

Optimization of sodium triflinate concentration for PLIMB-mediated CF3 labeling of proteins: The generation of CF3 radicals in solution is dependent upon both PLIMB-mediated OH and NO2 radicals in-solution. The starting concentration of sodium triflinate in solution relative to both a fixed amount of H₂O₂, water (55 M) and PLIMB dose can be modified to optimize the levels of CF3 labeling for the downstream analysis of footprinting experiments. To further understand the effect of sodium triflinate concentrations on PLIMB-mediated CF3 radical generation, the baseline protein lysozyme was used with a fixed concentration of H₂O₂ (10 mM) and varying concentrations of sodium triflinate in solution. Optimization experiments led to the determination that 50 mM sodium triflinate was the optimal concentration for use in combination with a fixed dose of 10 mM hydrogen peroxide for PLIMB-mediated trifluoromethyl (•CF3) labeling.

### Example 5

### PLIMB-Mediated Lysozyme Footprinting in Variable Sodium Triflinate Concentrations

Experimental Conditions for optimization of sodium triflinate concentration in PLIMB-mediated CF3 footprinting: Solutions of lysozyme from chicken egg white were prepared in PBS buffer, pH 7.4, with various concentrations of sodium triflinate (50 μM, 10 mM, 50 mM, 150 mM or 500 mM) and 10 mM H₂O₂ in the reaction mixture. Samples from each solution were prepared in 50 uL replicate aliquots and exposed to a fixed 75 second PLIMB dose. Following PLIMB exposure, samples were quenched with a 5 uL solution of 250 mM methionine in PBS, pH 7.4. They were then digested and analyzed by LC-MS/MS/MS.

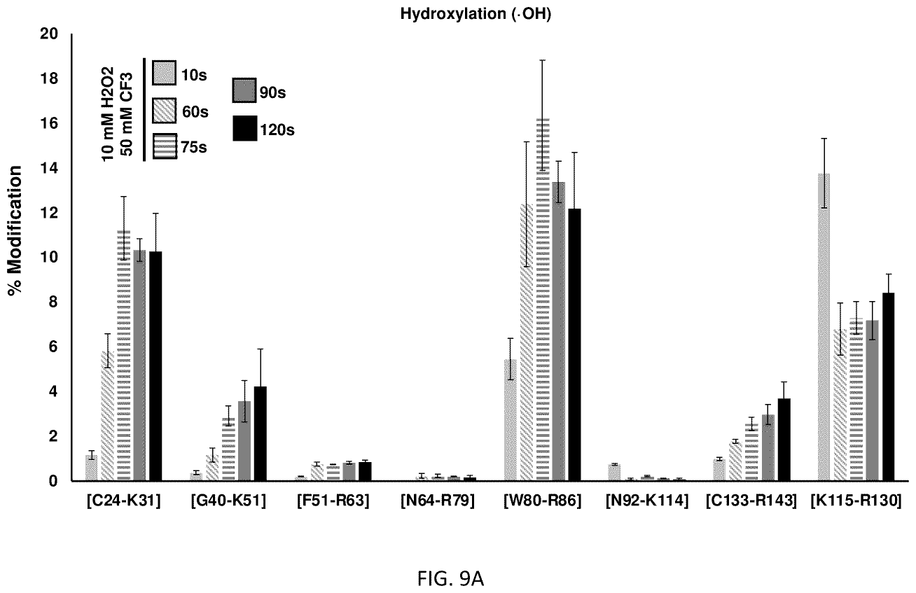

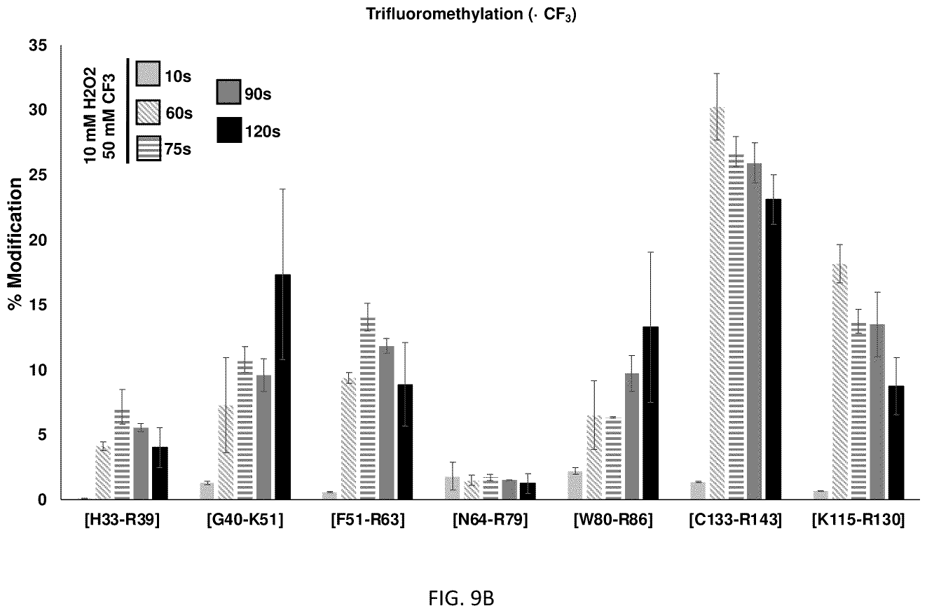

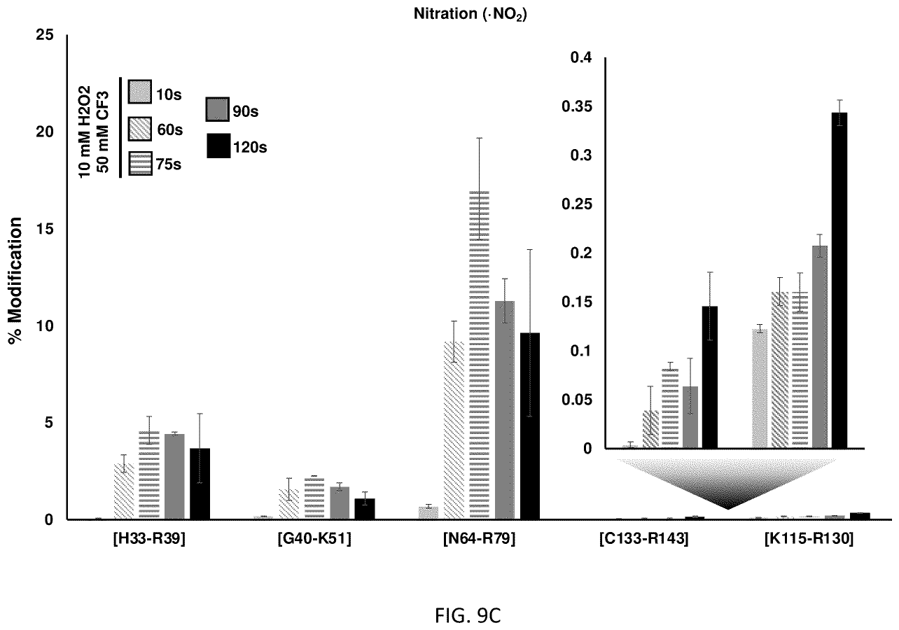

Results: After mass spectrometry analysis, a peptide level-analysis of lysozyme was performed across reaction conditions, which allowed for quantification and characterization of percent PLIMB-mediated CF3 radical labeling as a function of sodium triflinate concentrations (FIG. 9). The lowest concentration of sodium triflinate tested, 50 uM, yielded very low levels of PLIMB-mediated CF3 modification of lysozyme peptides (>1%). The optimal concentration of sodium triflinate for CF3 labeling in combination with 10 mM hydrogen peroxide was observed at 50 mM, with 5-25% modification of peptide. High concentrations of sodium triflinate in-solution (150 and 500 mM) produced higher levels of variability, likely due to solubility issues and downstream sample-handling.

### Example 6

### Optimization of Buffer Composition for PLIMB-Mediated CF3 Labeling

To consistently generate and label proteins in solution using the PLIMB platform, a precise understanding and control of the reaction conditions is essential. In particular, the intrinsic oxidative scavenging potential of buffer components present in solution may yield a different concentration of radical species in solution. In addition to buffers themselves acting as radical scavengers, the reaction environment, i.e., the various radical species generated and their corresponding concentrations, may be drastically different due to pH changes, concentration of radicals, radical-radical interactions, radical propagation, and half-lives. Therefore, aside from the generation of the radical species, the fate of the radical species themselves should be considered across different buffer components. For example, a radical may be generated and label the protein, interact and bond with another radical, interact with a precursor (i.e. sodium triflinate, water or the buffer) to create more radicals, or become inactive due to their respective half-lives. Due to many of its desirable properties, PBS is the most commonly used buffer in footprinting experiments. However, many biopharmaceutical companies and biochemical researchers use “Goods buffers,” such as HEPES, MES and PIPES in solution to mimic the neutral biological pH that proteins inhabit in vivo. The aforementioned Goods buffers have been shown to effectively scavenge OH radicals in solution. (Grady et al., 1988) However, in the presence of sodium triflinate, a different reactivity environment exists, and hence, the potential for scavenging, inadvertent radical generation, and inadvertent reactions exists. The buffers may also form radical species themselves when exposed to plasma, like that used for PLIMB, therefore different species may be present in PLIMB compared to other footprinting approaches that primarily utilize photolysis. Overall, a need exists for the investigation of radical generation in solution by plasma for optimal CF3 labeling. Herein, optimization indicates that both the PBS and HEPES buffers are suitable for PLIMB-mediated trifluoromethyl (•CF3) labeling.

### PLIMB-Mediated Lysozyme CF3 Footprinting in PBS Vs HEPES Buffer

Experimental conditions: Solutions of lysozyme (from chicken egg white) were prepared in PBS buffer, pH 7.4 or HEPES buffer, pH 6.0 with the trifluoromethyl-footprinting reagents (10 mM H2O2 and 50 mM sodium triflinate) present in the reaction mixture. Samples from each solution were prepared in 50 uL replicate aliquots and exposed to a fixed 75-second PLIMB dose. Following PLIMB exposure, samples were quenched with a 5 uL solution of 250 mM methionine in PBS, pH 7.4, and then digested and analyzed by LC-MS/MS.

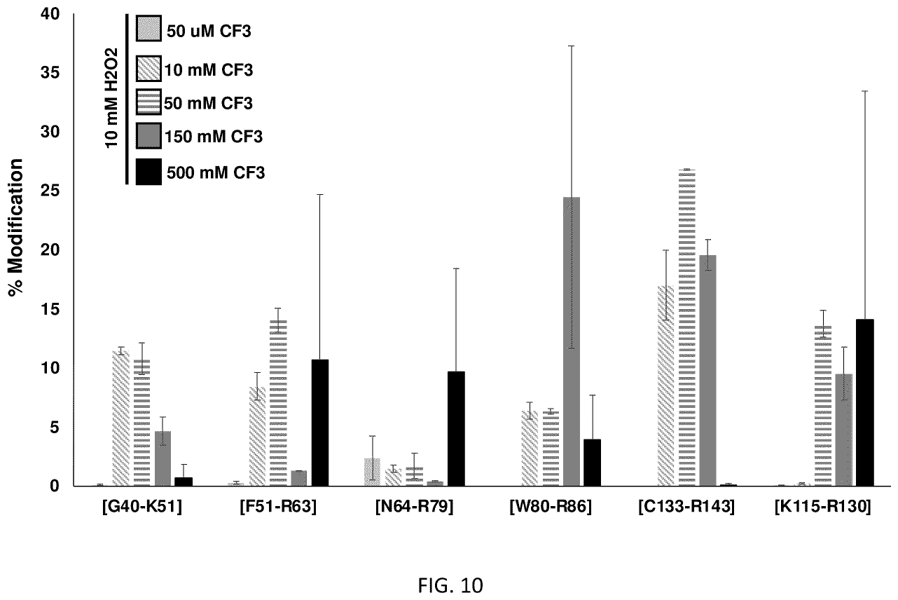

Results: After mass spectrometry analysis, a peptide-level analysis of lysozyme was performed across reaction conditions, which allowed for quantification and characterization of percent PLIMB-mediated CF3 radical labeling as a function of buffer conditions (PBS, pH 7.4 vs HEPES, pH 6.0). The peptide-level analysis shows that after 75 seconds of PLIMB exposure, significant levels of modification are detected across every region of lysozyme, providing a basis for future footprinting analysis in either buffer condition (FIG. 10). There were no detected signs of global changes (increases or decreases) in modification between the protein in buffer conditions, indicating no significant disruption of secondary structure.

### Example 7

### Optimization of H₂O₂ for PLIMB-Mediated CF3 Footprinting

The generation and concentration of CF3 radicals in solution is dependent upon both PLIMB-mediated OH and NO2 radicals in-solution. As an additive, H₂O₂ (pKa 11.6) will inherently increase the oxidative potential of the reaction in water (pKa 14.0) and may be beneficial for CF3 radical production. A lower pKa means species are more likely to give up their protons/electrons and be subject to subsequent oxidative events for radical generation. Furthermore, the peroxide is not necessary, but will speed up radical generation because for each water lysis event. Without the addition of peroxide one OH radical is produced from one water molecule, whereas two hydroxyl radicals are produced per molecule of peroxide. Therefore more downstream CF3 radicals produced from the sodium triflinate precursor when peroxide is added. Therefore, the starting concentration of H₂O₂ in solution relative to both a fixed amount of sodium triflinate, water (55 M) and a fixed PLIMB dose can be modified to optimize the levels of CF3 radicals produced in solution for downstream labeling in footprinting experiments. The amount of CF3 radicals produced using a fixed PLIMB dose and hydrogen peroxide are currently unknown. Here, to further understand the effect of starting H₂O₂ concentration (0-100 mM) on PLIMB-mediated CF3 radical generation, the baseline protein lysozyme was used with a fixed concentration of sodium triflinate (50 mM) in solution. Overall, this optimization led to the conclusion that the optimal concentration of hydrogen peroxide for PLIMB-mediated trifluormethyl (•CF3) labeling using 50 mM sodium triflinate was 10 mM.

### Example 8

### Optimization of Peroxide Concentration for PLIMB-Mediated CF3 Labeling of Lysozyme

Experimental Conditions: Solutions of lysozyme (from chicken egg white) were prepared in PBS buffer, pH 7.4 with various concentrations of H2O2 (0, 10, or 100 mM) and a fixed 50 mM sodium triflinate concentration in solution. Samples from each solution were prepared and 50 L replicate aliquots were exposed to a fixed 75-second PLIMB dose. Following PLIMB exposure, samples were quenched with a 5 uL solution of 250 mM methionine in PBS, pH 7.4. They were then digested and analyzed by LC-MS/MS.

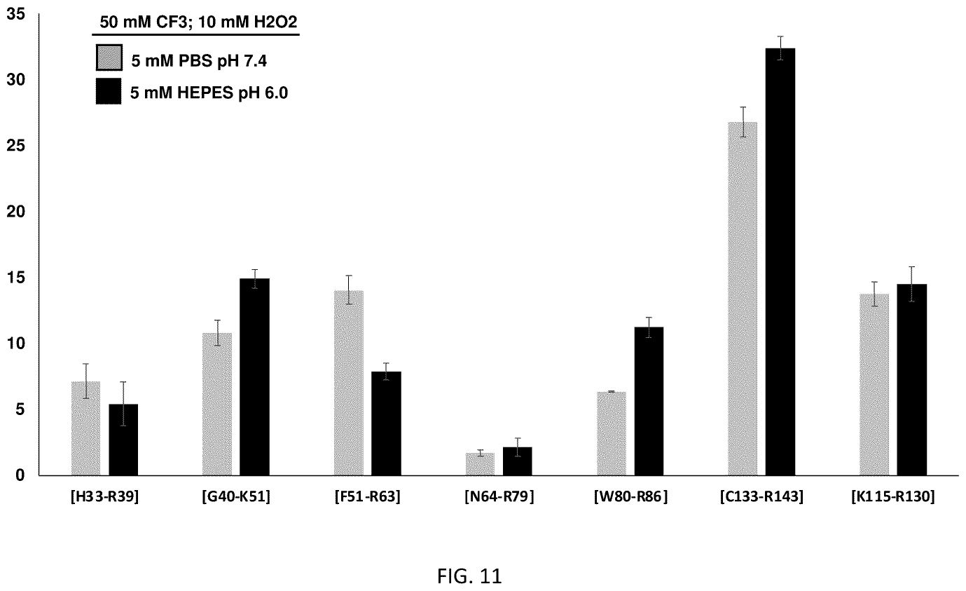

Results: After mass spectrometry analysis, a peptide level-analysis of lysozyme was performed across reaction conditions, which allowed for quantification and characterization of percent PLIMB-mediated CF3 radical labeling as a function of H2O2 concentrations (FIG. 11). Without the presence of peroxide in solution, the level of CF3 modification of peptides was very low <2%, whereas concentrations of 10 and 100 mM H2O2 in solution produced higher levels of CF3 modification (generally 5-25%) (FIG. 11). However, conditions utilizing 100 mM H2O2 occasionally led to lower levels of CF3 modification—likely due to solubility issues and/or a favoring of OH labeling over CF3.

### Example 9

### Optimization and the Novel Use of Tert-Butyl Hydroperoxide (TBHP) for Simultaneous PLIMB-Mediated Hydroxyl (•OH), Nitro (•NO2) and Trifluoromethyl (•CF3) Footprinting of Protein

While previous footprinting approaches such as FPOP have routinely utilized photolysis of H2O2 in solution for radical generation, the use of TBHP in the protein footprinting reactions has yet to be demonstrated and holds great promise. Herein it is demonstrated for the first time PLIMB-mediated hydroxyl (•OH), Nitro (•NO2) and trifluoromethyl (•CF3) labeling in-solution utilizing sodium triflinate and TBHP additives. Given the complexity of radical species that may be formed in solution for PLIMB treatment of sodium triflinate and TBHP, the concentration for CF3 protein footprinting applications was assessed and optimized. Overall, this optimization led to the conclusion that 1 mM TBHP maximizes PLIMB-mediated hydroxyl (•OH), Nitro (•NO2) and trifluoromethyl (•CF3) labeling. Moreover, TBHP appeared to increase labeling in comparison to H2O2, for PLIMB-mediated radical CF3 labeling across all concentrations.

### Example 10

### PLIMB-Mediated Lysozyme CF3 Footprinting in Presence and Absence of Various Concentrations of TBHP and H₂O₂

Experimental Conditions for PLIMB-mediated CF3 footprinting in presence and absence of TBHP: Solutions of lysozyme (from chicken egg white) were prepared in HEPES buffer, pH 6.0 with various concentrations of TBHP or H₂O₂ (0, 1, 5, 10, 50, or 100 mM) and 50 mM sodium triflinate in the reaction mixture. Samples from each solution were prepared in 50 uL replicate aliquots and exposed to a fixed 75-second PLIMB dose. Following PLIMB exposure, samples were quenched with a 5 uL solution of 250 mM methionine in PBS, pH 7.4. They were then digested and analyzed by LC-MS/MS.

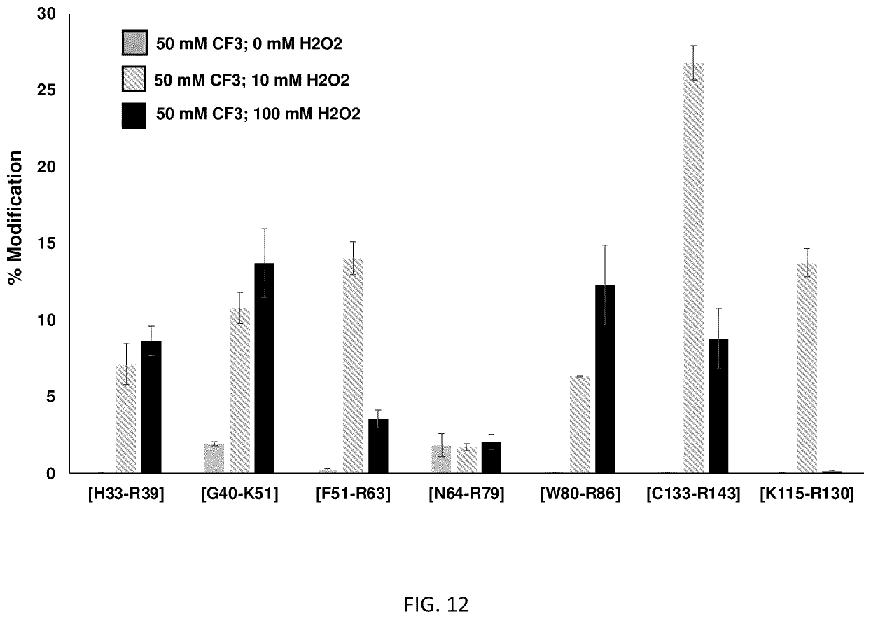

Results: After mass spectrometry analysis, a peptide-level analysis of lysozyme was performed across reaction conditions, which allowed for quantification and characterization of percent PLIMB-mediated radical labeling as a function of TBHP and H₂O₂ concentration (0-100 mM) (FIG. 12). Without the presence of peroxide in solution with 50 mM sodium triflinate, the level of CF3 modification of peptides was very low: <1%. However, a high level of trifluoromethyl (•CF3) labeling was detected for all peptides with TBHP (>5%) vs H2O2 (<5%). Notably, PLIMB-mediated CF3 labeling appeared to be maximized with the lowest concentration (1 mM) of TBHP. Additionally, it appears that lower concentrations of TBHP are needed in solution and the additive outperformed H2O2 for maximal levels of CF3 peptide modification in all cases.

### Example 11

### Optimization of Electrode Position for Dual, Simultaneous PLIMB-Mediated Hydroxyl (•OH), Nitro (•NO2) and Trifluoromethyl (•CF3) Labeling of Lysozyme

Hydroxyl radical generation and the dose mediated by the PLIMB electrode is highly dynamic. The PLIMB electrode exists at the interface of the air and solution and may produce different species of radicals in-solution for downstream labeling depending on the electrode distance.

Experimental Conditions for optimization of electrode position for PLIMB-mediated footprinting: Solutions of lysozyme (from chicken egg white) were prepared in PBS buffer, pH 7.4, with 10 mM H2O2 and 50 mM sodium triflinate in 50 uL replicate aliquots. Three replicates were exposed to a fixed 60-second PLIMB dose. Following PLIMB exposure, samples were quenched with a 5 uL solution of 250 mM methionine in PBS, pH 7.4. Samples were then digested and analyzed by LC-MS/MS.

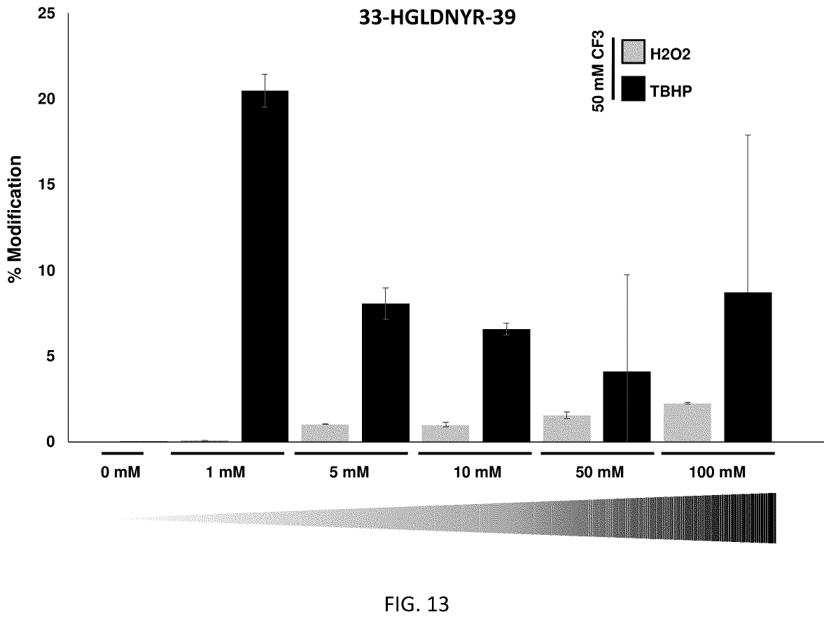

Results: After mass spectrometry analysis, a peptide level-analysis of lysozyme was performed across reaction conditions, which allowed for quantification and characterization of percent PLIMB-mediated radical labeling as function of electrode position (submersed vs suspended). A high level of hydroxyl (•OH). Nitro (•NO2) and trifluoromethyl (•CF3) labeling was detected for all peptides with both the submersed and suspended electrode (FIG. 13). Radical labeling appeared to be maximized in the suspended electrode position opposed to the immersed electrode for all modifications. Interestingly, the level of Nitro (•NO2) modification appeared to be most dependent on electrode position, with <1% labeling observed for the submersed electrode and 1.5-4% for the suspended electrode. Overall, this optimization led us to conclude that the suspended electrode position compared to submerged appears to maximize PLIMB-mediated hydroxyl (•OH), Nitro (•NO2) and trifluoromethyl (•CF3) labeling for future footprinting experiments.

### Example 12

### Optimization of Dual, Simultaneous PLIMB-Mediated Hydroxyl (•OH), Nitro (•NO2) and Trifluoromethyl (•CF3) Labeling of Protein in the Presence of Radical Scavengers

The presence of common biological additives histidine, DTT, TCEP, lipids, sucrose in a protein solution that are known to be OH radical scavengers such as histidine, DTT. TCEP, lipids, sucrose, may be detrimental to footprinting experiments and significantly reduce the amount of subsequent modification. Additionally, if the amount of scavenger present between two samples differs, the downstream data may be very difficult to compare, since the overall levels of modification will be artificially affected by the relative concentration of scavenger(s). If an understanding of scavenging potential is ascertained, it may be possible to “match” and optimize the resulting radical dose between samples with complex matrices. The influence of radical scavengers on plasma-generated radicals in solution and the resulting reactive environment has not yet been reported on. Many scavengers cannot be avoided in protein footprinting experiments, especially in the investigation of protein-drug binding/interactions. For example, DMSO is the baseline solvent for preparing stock solutions of small molecule compounds for drug discovery and screening, and the PLIMB platform may be optimized to accommodate for labeling in these scavenging conditions. Here, the baseline protein lysozyme was used to optimize and assess the PLIMB-mediated radical footprinting of protein in the presence of the radical scavenger DMSO. Overall, this optimization led to the conclusion that the addition of radical scavengers, such as DMSO to the reaction mixture leads to a decrease, but not detrimental loss of labeling in PLIMB-mediated footprinting.

PLIMB-mediated OH-footprinting of myoglobin in the absence and presence of DMSO with varying concentrations: The goal of this study was to determine the influence and effects of baseline DMSO concentrations on PLIMB labeling in hydroxyl radical footprinting experiments. The approach was to measure the amount of PLIMB-mediated hydroxyl radical labeling of a baseline protein, myoglobin, in five different reaction conditions (0, 0.5, 1.0, 1.5 and 2.0% DMSO [V/V]).

Experimental Conditions for optimization of PLIMB-mediated footprinting in the absence and presence of DMSO with varying concentrations: Several solutions of myoglobin were prepared with 0, 0.5, 1.0 or 2.0% DMSO (V/V) present in the reaction mixture. Samples from each solution were prepared in 50 L volumes by aliquoting and were exposed to a fixed PLIMB dose for 20 seconds in three replicates. Following PLIMB exposure, samples were quenched with a 50 L solution of 25 mM methionine in pH 7.4 PBS. They were then digested and analyzed by LC-MS/MS.

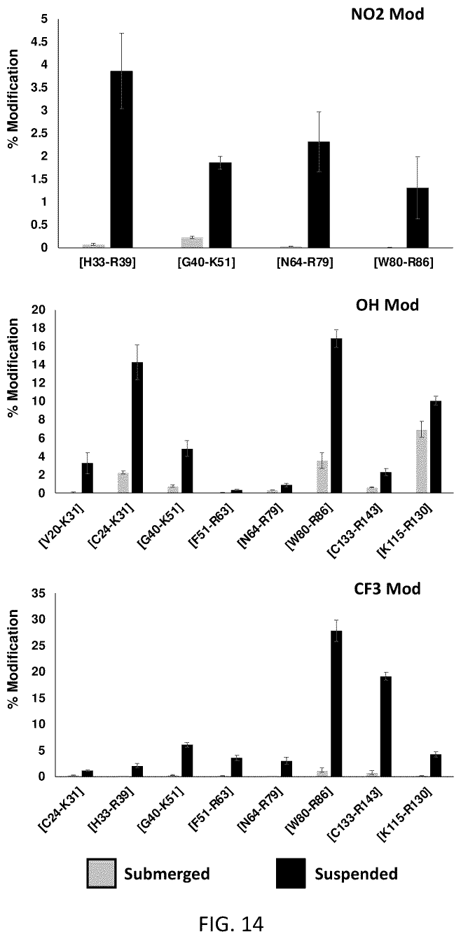

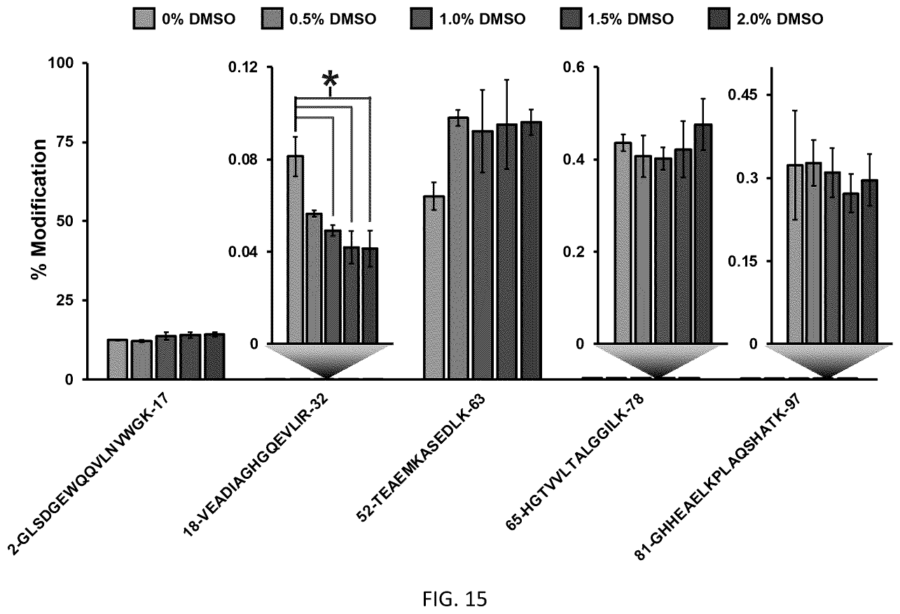

Results: After mass spectrometry analysis, a peptide-level analysis was performed, which allowed for quantification and characterization of labeling in the absence or presence of low levels of DMSO in the reaction mixture when exposed to PLIMB. FIGS. 14-15 and Table 1 show the average percent modification under each condition, with an asterisk indicating a significant change in level of modification. The peptide-level analysis shows that after 20 seconds of PLIMB exposure, significant levels of modification are detected across most regions of protein, providing a basis for future footprinting analysis in the presence of DMSO. There were no detected signs of global changes (increases or decreases) in modification between the protein in conditions containing DMSO, indicating no significant disruption of secondary structure. The consistent modification of these peptides shows a relatively equivalent oxidative potential of the protein in each condition. A t-test was performed to quantitatively compare any significant differences in the degree or percent modification of myoglobin peptides between conditions with and without DMSO. The conditions that showed statistical significance are shown in bold below, along with the % modification. Myoglobin peptides [18-32], [100-119], [135-146] and [147-154] showed statistically significant decreases (˜8-49%) in PLIMB-mediated hydroxyl modification with DMSO present in the reaction mixture relative to samples without the additive present (FIG. 14, FIG. 15, Table 1). The progressive decrease in labeling with increasing DMSO concentrations of peptides [18-32], [135-146] and [147-154] suggests minor, but not detrimental scavenging of radicals in the PLIMB reaction conditions. All the peptides where a decrease was observed maintained a sufficient level of detectable labeling. Interestingly, a few peptides [2-17], [52-63] and [120-134] showed a slight increase in hydroxyl radical labeling, likely due to DMSO interactions with the heme ligand¹ of myoglobin.

TABLE 1
Percent PLIMB-mediated Modification of
Myoglobin [2-154].

| Detected Myoglobin | 0% | 0.5% | 1.0% | 1.5% | 2.0% |
|---|---|---|---|---|---|
| Peptide | DMSO | DMSO | DMSO | DMSO | DMSO |
| 2-GLSDEWQQVLNVWGK- | 12.45 | 12.15 | 13.65 | 14.03 | 14.30 |
| 17 (SEQ ID NO: 1) | | | | | |
| 18-VEADIAGHGQEVLIR- | 0.08 | 0.06 | 0.05 | 0.04 | 0.04 |
| 32 (SEQ ID NO: 2) | | | | | |
| 52-TEAEMKASEDLK-63 | 57.85 | 82.60 | 78.50 | 80.57 | 81.23 |
| (SEQ ID NO: 3) | | | | | |
| 65-HGTVVLTALGGILK- | 0.44 | 0.41 | 0.40 | 0.42 | 0.48 |
| 78 (SEQ ID NO: 4) | | | | | |
| 81-GHHEAELKPLAQSHA | 0.32 | 0.33 | 0.31 | 0.27 | 0.30 |
| TK-97 (SEQ ID NO: | | | | | |
| 5) | | | | | |
| 100-IPIKYLEFISDAII | 95.35 | 87.07 | 89.13 | 92.40 | 88.23 |
| HVLHSK-119 (SEQ ID | | | | | |
| NO: 6) | | | | | |
| 120-HPGDFGADAQGAMT | 51.05 | 61.95 | 71.00 | 65.43 | 66.90 |
| K-134 (SEQ ID NO: | | | | | |
| 7) | | | | | |
| 135-ALELFRNDIAAK- | 0.72 | 0.27 | 0.32 | 0.33 | 0.36 |
| 146 (SEQ ID NO: 8) | | | | | |
| 147-YKELGFQG-154 | 11.00 | 8.76 | 7.65 | 7.70 | 5.75 |
| (SEQ ID NO: 9) | | | | | |

After performing hydroxyl radical protein footprinting with PLIMB, percent modification of peptides across the entire myoglobin sequence was calculated for the different experimental conditions: 0, 0.5, 1.0, 1.5 and 2.0% DMSO (v/v). T-tests were performed to detect statistically significant differences in modification between conditions. Colored cells indicate a significant decrease in percent modification across conditions (p<0.05). Green cells represent an increase in modification relative to the DMSO-free condition and red cells represent a decrease.

### Example 13

### Optimization of pH Conditions for Simultaneous PLIMB-Mediated Hydroxyl (•OH), Nitro (•NO2) and Trifluoromethyl (•CF3) Labeling of Protein

The nature and abundance of reactive oxygen and nitrogen species produced by a plasma discharge at the gas-liquid interference include but are not limited to (O3, H2O2, H3O+, NO2-, and NO3⁻) and short-lived radical species (•OH, •O2-, HOO•, and •NO2). It should be noted that for every instance of homolysis and oxidation of water or other additives in solution occurs, whether via radiolysis, photolysis, or a plasma discharge, Lewis acids are created and can rapidly decrease the pH. (Judée et al., 2018; Ryu et al., 2013) Moreover, as previously stated, the abundance of reactive species and radicals present in solution is highly dependent on pH and determines what reactions are more likely to subsequently occur. For example, in the process of CF3 radical production, CF3 from sodium triflinate is displaced by OH radicals in solution and the conjugate base sulfurous acid is produced in solution. Similarly, each time H2O (pKa˜14) is lysed by plasma in solution, a corresponding (•H) and (—HO), radical is produced and can produce (H2O2) (pKa˜11.5). Nitrogen gas (N2) is also highly abundant in the atmosphere and is readily oxidized to nitrogen dioxide (NO2), which, upon photolysis or treatment with PLIMB, may yield a variety of reactive NOx species, including nitrate (NO3-) and subsequently nitro radicals (•NO2). Therefore, a plethora of potential reaction landscapes exist, and as a result, the pH of solution during these radical reactions can be monitored to tune reaction efficacy. Indeed, trifluoromethyl radical reactions may be highly sensitive and dependent on initial solution pH and buffering.

Aside from tuning reaction conditions for optimal labeling, a concern exists for the subsequent denaturation of proteins in solution, which commonly occurs between pH 2-5. Accordingly, experiments were conducted to optimize the pH for the simultaneous PLIMB-mediated hydroxyl (•OH), Nitro (•NO2) and trifluoromethyl (•CF3) labeling of protein in-solution to maintain a biologically relevant pH and resist any changes. Here, pH was monitored after various plasma doses and it was determined that the optimal buffer conditions to offset any changes occurring due to production of various reactive species in solution during simultaneous hydroxyl (•OH), Nitro (•NO2) and trifluoromethyl (•CF3) labeling of protein was 50 mM PBS, pH 7.4.

### Example 14

### Optimization of Additives and Buffer for Conservation of In-Solution pH after Plasma Treatment for PLIMB-Mediated OH, CF3 and NO2 Footprinting

Experimental Conditions for evaluation of pH in PLIMB-mediated OH, CF3 and NO2 footprinting: Solutions of “baseline” OH labeling (5 mM PBS, pH 7.4) and “baseline” CF3/trifluoromethyl labeling conditions (10 mM H2O2, 50 mM sodium triflinate in PBS, pH 7.4) were prepared in 50 L aliquots. Solutions of CF3/trifluoromethyl labeling conditions with less H2O2 (5 mM H2O2, 50 mM sodium triflinate, 5 mM PBS (pH 7.4), in the absence of H2O2 (50 mM sodium triflinate, 5 mM PBS (pH 7.4), and in the absence of sodium triflinate (10 mM H2O2, 5 mM PBS (pH 7.4) were also prepared. Additionally, solutions of baseline CF3/trifluoromethyl labeling conditions (10 mM H2O2, 50 mM sodium triflinate) were prepared in buffer conditions including 5 mM HEPES (pH 6.0), 10 mM HEPES (pH 6.0), 20 mM HEPES (pH 6.0), 50 mM HEPES (pH 6.0), 5 mM HEPES (pH 7.4), 50 mM PBS (pH 7.4) and 100 mM PBS (pH 7.4). Samples from each solution were aliquoted in 50 uL volumes in PCR tubes and exposed to varied PLIMB doses (0, 5, 10, 20, 40 or 60 seconds). The initial and resulting pH after PLIMB treatment was measured by applying a small aliquot of sample to pH paper.

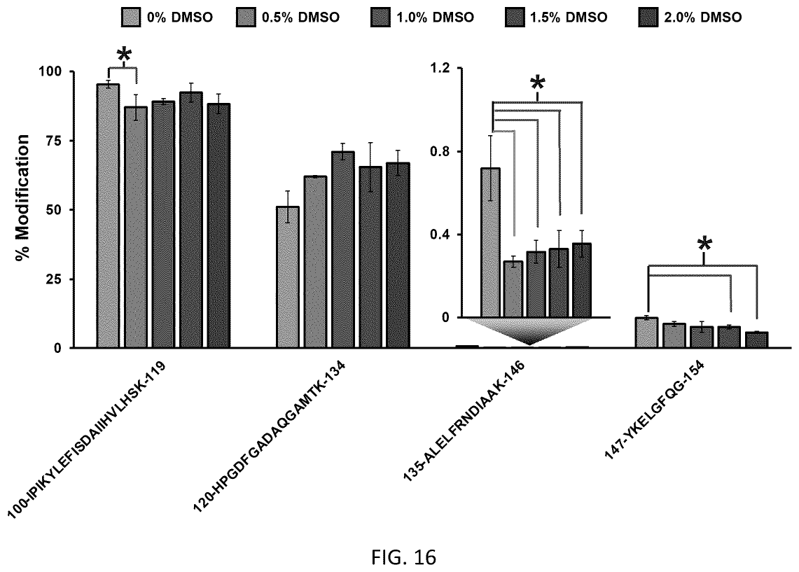

### Results

[pH Optimization 1]. In the first optimization and evaluation of solution pH after plasma treatment, baseline conditions were utilized. The resulting solution pH of the baseline OH labeling conditions (5 mM PBS, pH 7.4) treated with plasma was reduced at a ˜40-second dose, whereas in the baseline CF3/trifluoromethyl labeling conditions (10 mM H2O2, 50 mM sodium triflinate in PBS, pH 7.4), the pH was reduced at just 5 seconds of plasma treatment (FIG. 16). The pH was reduced at a much lower plasma dose in the baseline trifluoromethyl footprinting conditions, likely because the reactive environment is much more complex and more reactive species are produced with the sodium triflinate and hydrogen peroxide additives in the trifluoromethyl footprinting conditions.

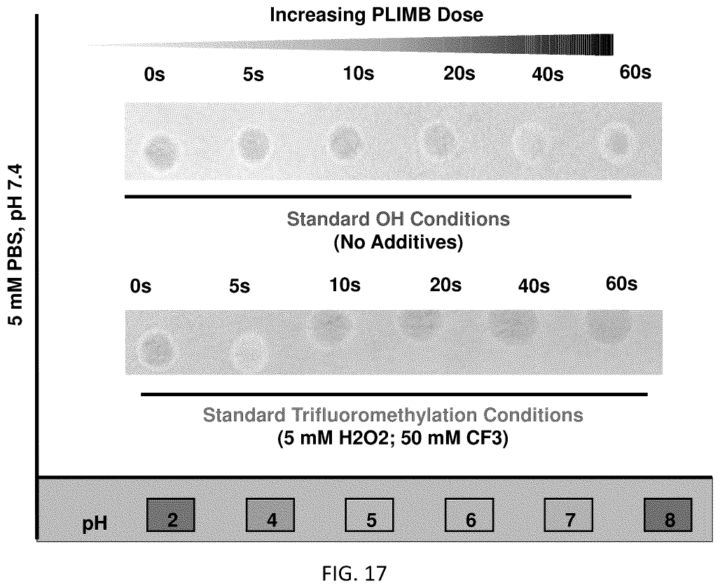

pH Optimization 2. Next, in the second optimization the influence of each additive on the baseline trifluoromethyl footprinting conditions after plasma treatment was investigated (FIG. 17). Interestingly, the pH of trifluoromethyl labeling conditions with a lower concentration of H2O2 (5 mM H2O2, 50 mM sodium triflinate, 5 mM PBS (pH 7.4) or in the absence of H2O2 (50 mM sodium triflinate, 5 mM PBS (pH 7.4) were acidic upon the same 5-second plasma dose that the baseline conditions were. Therefore, H2O2 as an additive appeared to have a minimal effect on the reactive environment and solution pH after plasma treatment. However, in the absence of the sodium triflinate additive (10 mM H2O2, 5 mM PBS (pH 7.4), the reaction pH was maintained even at a 20-second plasma dose. In this optimization, the concentration of the sodium triflinate additive was influential when assessing pH of solution after plasma treatment, and the initial buffer conditions should be tuned accordingly if the pH is to be maintained during plasma treatment.

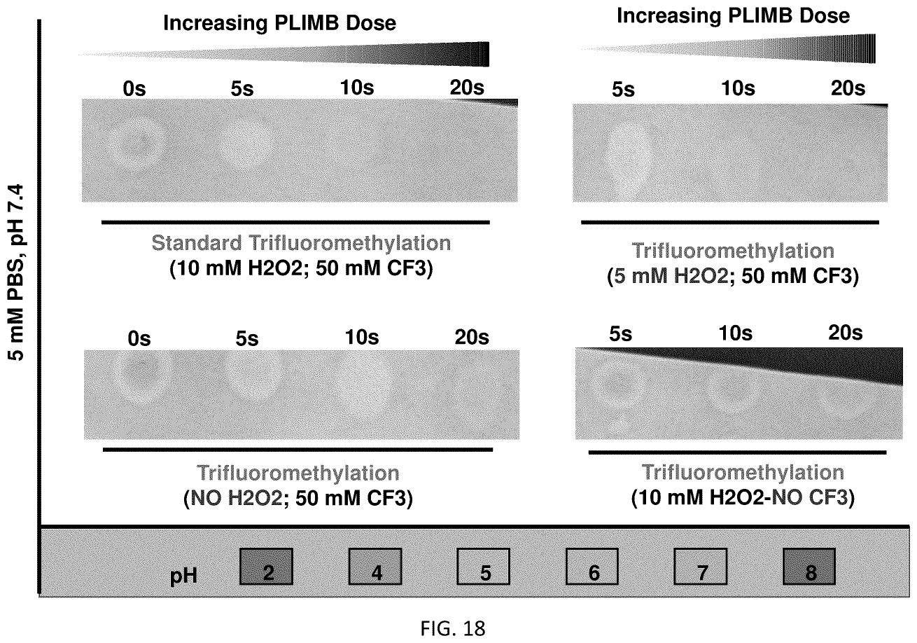

pH Optimization 3. Next, in the third optimization, the influence of utilizing a different biologically relevant buffer (HEPES) and buffer capacity on the baseline trifluoromethyl footprinting conditions after plasma treatment was evaluated (FIG. 18). HEPES buffer was used at pH 6 at an equivalent molarity as PBS utilized in baseline trifluoromethyl footprinting and observed an identical drop in pH at just 5 seconds of plasma treatment. Increasing the molarity and buffering capacity of HEPES to 10 mM, 20 mM and 50 mM had a negligible effect on maintaining solution pH after plasma treatment.

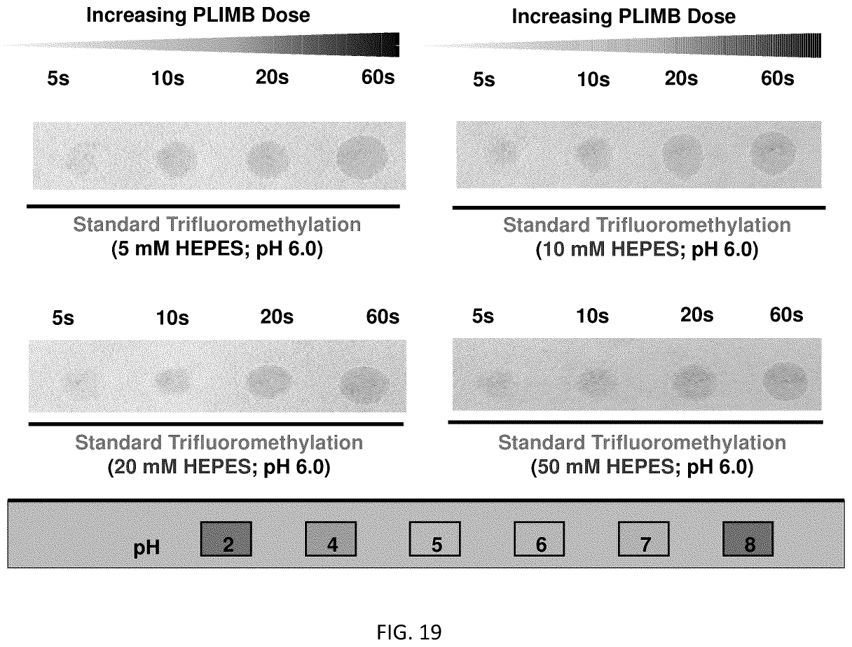

pH Optimization 4. In the fourth optimization, the influence of utilizing HEPES buffer corrected to the same starting pH (7.4) as the PBS utilized in baseline trifluoromethyl footprinting conditions after plasma treatment was evaluated (FIG. 19). The pH of HEPES (pH 7.4) was maintained for up to a 10 seconds of plasma dose before becoming acidic, suggesting it may be a better alternative to PBS for maintaining solution pH. Lastly, the influence of buffering capacity for the baseline 5 mM PBS buffer, pH 7.4, utilized in footprinting and observed both 50 mM PBS and 100 mM improved and maintained solution pH during plasma treatment up to 20 s (FIG. 19).

### Example 15

### PLIMB-Mediated Lysozyme CF3 Footprinting in Presence and Absence of Transition Metal Catalysts

Optimization of transition metals catalysts for PLIMB-mediated OH, NO2 and CF3 footprinting: Transition metal catalysts are redox reactive species and have commonly been added to radical-based photo and electrochemical reactions to shuttle electrons and act as reaction intermediates. Historically, Fe (II) in solution with hydrogen peroxide has been shown as an effective mechanism to generate OH radicals and promote the catalytic oxidation of tartaric acid. (Fenton, 1894) However, Fenton chemistry has been shown to be less than ideal for protein footprinting and HOS analysis, due to the long lifetime of radical species produced. In this optimization, H2O2 was intentionally left out of reactions to avoid competition or a more Fenton-like chemical reaction pathway.

Experimental conditions for PLIMB-mediated footprinting in absence and presence of transition metals: Solutions of lysozyme (from chicken egg white) were prepared in PBS buffer, pH 7.4 in the presence and absence of 2.5 mM copper sulfate or iron sulfate and 50 mM sodium triflinate in 50 uL replicate aliquots. One sample in baseline trifluoromethyl conditions was prepared (10 mM H2O2, 50 mM sodium triflinate). Three replicates were exposed to a fixed PLIMB doses (75 s). Following PLIMB exposure, samples were quenched with a 5 uL solution of 250 mM methionine in PBS, pH 7.4. Samples were then digested and analyzed by LC-MS/MS.

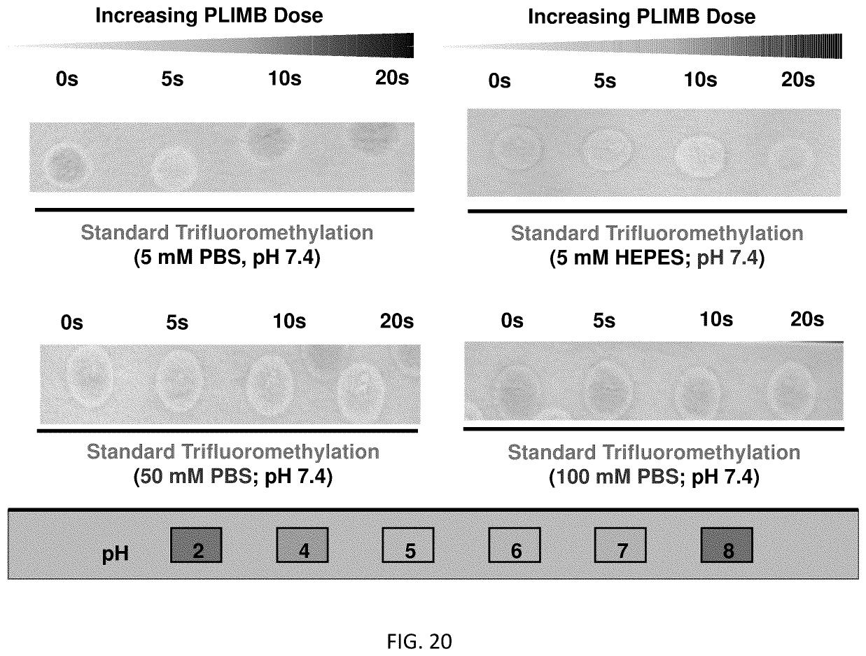

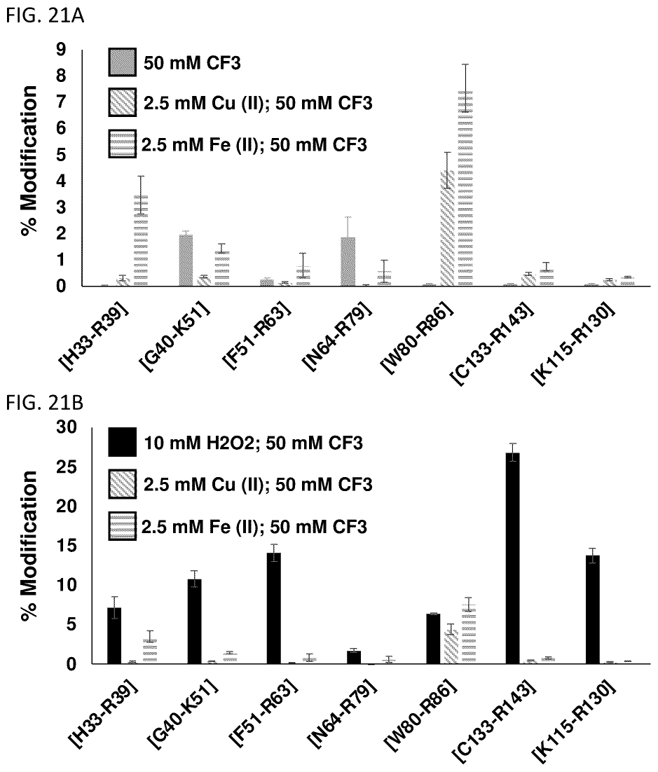

Results: After mass spectrometry analysis, a peptide-level analysis of lysozyme was performed, which allowed for quantification and characterization of CF3 labeling in the presence or absence of low the transition metal catalysts (Cu II and Fe II) in the reaction mixture. Following PLIMB exposure, levels of CF3 modification were detected across most regions of lysozyme with the sodium triflinate reagent and with or without the metal additives (FIG. 20). In the absence of H2O2, with sodium triflinate in solution (50 mM) low levels of CF3 modification (<1-2%) across the lysozyme peptides were observed. Overall, addition of Cu II and Fe II appeared to improve CF3 labeling with levels of <1-7% observed across the lysozyme peptides. However, in comparison to the baseline trifluoromethyl footprinting conditions (10 mM H2O2, 50 mM CF3), CF3 labeling ranged from 2-25% and was much higher with sodium triflinate than with the transition metal additives alone. Overall, this optimization indicates that the addition of transition metal catalysts to the reaction mixture did not benefit the level of CF3 labeling in comparison to H2O2.

### Example 16

### Fluorescent Dye (BODIPY) to Measure CF3 Radical Concentrations

Protein footprinting requires equal radical doses of samples to ensure accurate data interpretation. Sometimes, samples of different chemical compositions cannot be avoided and thus, achieving the optimal effective dose, i.e., the extent of radical modification that the target biomolecule experiences after a given exposure to radicals, between samples would benefit from a quick and simple measurement technique. A chemical reporter with a measurable output once modified with CF3, such as fluorescence, can be utilized for effectively monitoring and delivering equal PLIMB doses across samples. In particular, a chemical variant of boron dipyrromethene (BODIPY) has promise for this application. Singly and doubly-CF3-modified BODIPY exhibits a shift in fluorescence emission wavelength of 10-20 nanometer over its unmodified chemical structure. Here, BODIPY can be used for monitoring and optimization of PLIMB dose and thus. CF3 radical production. A dose normalization between replicate samples can be performed by measuring CF3 radical production in real time with BODIPY and exposure times will be modulated to achieve exact doses for each replicate sample.

### Example 17

### Radical Generation Rate and Dose: Power Source, Electrode Composition/Geometry and Feed Gas

In addition to the treatment time and distance from the electrode to the sample, the applied power source, electrode material, geometry, and feed gas are variables to consider in optimization and radical generation. PLIMB has primarily been operated with surrounding air and a stainless steel or tungsten electrode with an applied voltage. The PLIMB system produces pulsed bursts of hydroxyl radicals in solution. The way the initial voltage is applied, therefore has an effect on the downstream species and concentrations of radicals produced. Varying the applied voltage, pulse length and waveshape may therefore change the amounts and species of radicals present in an advantageous way. Additionally, in applications outside of footprinting, the use of different electrode materials, including copper and platinum, as well as feed gases such as argon, helium, nitrogen, and carbon dioxide may change the downstream reactive species produced. While many reactive oxygen and nitrogen species may be generated regardless of electrode material, geometry, and feed gas, optimizing these variables has the potential for fine tunability of reaction chemistry and the resulting reactive radical species. Here, the effect of different electrode materials, geometry, and feed gas on downstream PLIMB-mediated multiplexed labeling of proteins can be optimized. Tetrafluoromethane (CF4) gas in the feed gas may be used to react with high energy electrons and oxygen to produce CF3 radicals.

### Example 18

### Protein Sizes, Total Protein Concentration and Solution Composition (in the Presence and Absence of Drugs and mAbs)

Protein footprinting requires equal radical doses and the correct controls in place to ensure accurate data interpretation. The effect and influence of protein size, amino-acid composition and structure on downstream scavenging and the resulting labeling can be optimized. For example, differences in labeling with a fixed PLIMB dose for an antigen (such as TNFa˜15 kDa) and an Infliximab (˜50 kDa Fab vs 150 kDa Mab) can be assessed. TNFα can be utilized in solution (from 1-1000 uM) and assess labeling and downstream footprinting to optimize concentration in the PLIMB-mediated multiplexed approach. Moreover, the radical scavenging potential of a solution may change if a drug, small molecule, or other protein species is present in one sample versus the other. For epitope mapping experiments a solution of the antigen may be labeled in the presence or absence of the antibody (bound vs. unbound). An internal standard for dosimetry may also be used, such as to ensure equal radical doses are delivered across samples with different scavenging potential. Different scavenging potentials (protein sizes, concentration and in the absence/presence of other drugs, Fabs and mAbs) may be used and the effect on downstream PLIMB-mediated multiplexed labeling of proteins can be assessed.

### Example 19

### Dual Carbene and Multiplexed Radical Footprinting

The resolution and coverage of PLIMB-mediated radical footprinting could be increased further with the addition of other labeling precursors. Diazirine reagents can be activated with ultraviolet light to generate carbene precursors and may be used for labeling. The PLIMB electrode generates both high energy electrons and light spanning the ultraviolet spectrum. As such, both PLIMB-mediated radical and carbene generated labels can be used to further increase the resolution of the footprinting approach described herein. Additionally, alternative additives to introduce the CF3 moiety may also be used to increase resolution.

### Example 20

### Optimization of PLIMB-Mediated Dual OH, NO2 and CF3 Footprinting Chromatography

The typical elution pattern of PLIMB-modified peptides on a standard reverse-phase C18 column is as follows: first OH, then NO2 and finally CF3. It may be desirable to decrease the complexity of the data produced by the CF3-modified peptides, where multiple isomeric peaks are often observed. For instance, there may be cases where data interpretation would be more straightforward if the products are collapsed down into a single peak for integration and quantification. The CF3 modification increases the hydrophobicity of peptides drastically and may be causing inadvertent column/sample interactions. In a broader sense, alternative forms of chromatography, such as hydrophobic interaction chromatography (HILIC) and ion mobility, in addition to alternative stationary phases, such as phenyl and FluoroSep-RP Phenyl, may yield advantageous differences in sample retention and elution profiles. Therefore, chromatography (e.g. HILIC, ion mobility and the fluorinated stationary phase) can be used for separation of the PLIMB modified peptides to further increase the utility of the footprinting approach provided herein.

### Example 21

### The Novel Use of TMAO for Preservation of Native Protein Structure in PLIMB-Mediated Footprinting

Triethylamine-N-oxide (TMAO) is a small molecule, kosmotrope, which stabilizes native, folded confirmations of proteins and prevents denaturation under stresses such as heat and pressure. TMAO may be used as a novel additive to prevent protein denaturation during radical footprinting experiments.

### REFERENCES

Borotto, N. B., Zhang, Z., Dong, J., Burant, B., & Vachet, R. W. (2017). Increased β-Sheet Dynamics and D-E Loop Repositioning Are Necessary for Cu(II)-Induced Amyloid Formation by β-2-Microglobulin. Biochemistry, 56(8), 1095-1104. https://doi.org/10.1021/acs.biochem.6b01198
Fenton, H. J. H. (1894). LXXIII.—Oxidation of tartaric acid in presence of iron. J. Chem. Soc., Trans., 65(0), 899-910. https://doi.org/10.1039/ct8946500899
Gau, B., Garai, K., Frieden, C., & Gross, M. L. (2011). Mass Spectrometry-Based Protein Footprinting Characterizes the Structures of Oligomeric Apolipoprotein E2, E3, and E4. Biochemistry, 50(38), 8117-8126. https://doi.org/10.1021/bi200911c
Gott, R. P., Engeling, K. W., Olson, J., & Franco, C. (2023). Plasma activated water: a study of gas type, electrode material, and power supply selection and the impact on the final frontier [10.1039/D2CP03489A]. Physical Chemistry Chemical Physics, 25(6), 5130-5145. https://doi.org/10.1039/D2P03489A
Grady, J. K., Chasteen, N. D., & Harris, D. C. (1988). Radicals from “Good's” buffers. Analytical Biochemistry, 173(1), 111-115. https://doi.org/https://doi.org/10.1016/0003-2697(88)90167-4
Hogan, J. M., Lee, P. S., Wong, S. C., West, S. M., Morishige, W. H., Bee, C., Tapia, G. C., Rajpal, A., Strop, P., & Dollinger, G. (2023). Residue-Level Characterization of Antibody Binding Epitopes Using Carbene Chemical Footprinting. Analytical Chemistry, 95(8), 3922-3931. https://doi.org/10.1021/acs.analchem.2c03091
Huynh, A. M., Menges, J., Vester, M., Dier, T., Huch, V., Volmer, D. A., & Jung, G. (2016). Monofluorination and Trifluoromethylation of BODIPY Dyes for Prolonged Single-Molecule Detection [https://doi.org/10.1002/cphc.201500869]. ChemPhysChem, 17(3), 433-442. https://doi.org/https://doi.org/10.1002/cphc.201500869
Jin, H., Zheng, N., Deng, W., & Chen, X. (2017). Research on the chemical reaction in CF4 plasma during fused silica processing.
Judée, F., Simon, S., Bailly, C., & Dufour, T. (2018). Plasma-activation of tap water using DBD for agronomy applications: Identification and quantification of long lifetime chemical species and production/consumption mechanisms. Water Research, 133, 47-59. https://doi.org/10.1016/j.watres.2017.12.035
Limpikirati, P., Pan, X., & Vachet, R. W. (2019). Covalent Labeling with Diethylpyrocarbonate: Sensitive to the Residue Microenvironment, Providing Improved Analysis of Protein Higher Order Structure by Mass Spectrometry. Analytical Chemistry, 91(13), 8516-8523. https://doi.org/10.1021/acs.analchem.9b01732
Minkoff, B. B., Blatz, J. M., Choudhury, F. A., Benjamin, D., Shohet, J. L., & Sussman, M. R. (2017). Plasma-Generated OH Radical Production for Analyzing Three-Dimensional Structure in Protein Therapeutics. Scientific Reports, 7(1). https://doi.org/10.1038/s41598-017-13371-7
Ryu, Y.-H., Kim, Y.-H., Lee, J.-Y., Shim, G.-B., Uhm, H.-S., Park, G., & Choi, E. H. (2013). Effects of Background Fluid on the Efficiency of Inactivating Yeast with Non-Thermal Atmospheric Pressure Plasma. PLoS ONE, 8(6), e66231. https://doi.org/10.1371/journal.pone.0066231
Walker, E. J., Bettinger, J. Q., Welle, K. A., Hryhorenko, J. R., Molina Vargas, A. M., O'Connell, M. R., & Ghaemmaghami, S. (2022). Protein folding stabilities are a major determinant of oxidation rates for buried methionine residues. Journal of Biological Chemistry, 298(5), 101872. https://doi.org/10.1016/j.jbc.2022.101872
Zhang, Y., Wecksler, A. T., Molina, P., Deperalta, G., & Gross, M. L. (2017). Mapping the Binding Interface of VEGF and a Monoclonal Antibody Fab-1 Fragment with Fast Photochemical Oxidation of Proteins (FPOP) and Mass Spectrometry. Journal of the American Society for Mass Spectrometry, 28(5), 850-858. https://doi.org/10.1007/s13361-017-1601-7

1. A method for multiplexed labeling of a biological molecule with trifluoromethyl and hydroxyl and/or nitro radicals, the method comprising:
a) providing a sample comprising two or more radical precursors and a biological molecule; and
b) generating a plasma in the sample, thereby converting the two or more radical precursors in the sample into trifluoromethyl and hydroxyl and/or nitro radicals which interact with the biological molecule, thereby labeling the biological molecule.

2. The method of claim 1, wherein the two or more radical precursors comprise sodium triflinate and hydrogen peroxide.

3. The method of claim 2, wherein the two or more radical precursors further comprise air.

4. The method of claim 2, wherein the sample comprises 1 nM to 4 M sodium triflinate and 1 nM to 10 M hydrogen peroxide, 50 uM to 500 mM sodium triflinate and 0.1 mM to 100 mM hydrogen peroxide, or 10 mM to 50 mM sodium triflinate and 10 mM to 100 mM hydrogen peroxide.

5.-7. (canceled)

8. The method of claim 1, wherein the two or more radical precursors comprise sodium triflinate and tert-butyl hydroperoxide (TBHP).

9. The method of claim 8, wherein the sample comprises 1 nM to 4 M sodium triflinate and 1 nM to 7.5 M TBHP, or 10 mM to 50 mM sodium triflinate and 0.1 mM to 100 mM TBHP.

10.-13. (canceled)

14. The method of claim 1, wherein the plasma is generated for 5 seconds to 120 seconds.

15. The method of claim 1, wherein plasma is generated from a plasma source point of a plasma electrode, wherein the plasma source point is placed within the sample, or wherein the plasma source point is placed at an interface of a gas and the sample.

16.-17. (canceled)

18. The method of claim 15, wherein the plasma source point is placed at a distance of 10 cm or less from the sample, or wherein the plasma source point is placed at a distance of 3 cm or less from the sample.

19. (canceled)

20. The method of claim 1, further comprising quenching the sample with a solution comprising a radical quencher following generating the plasma in the sample, wherein the radical quencher is methionine, cysteine, homocysteine, or taurine.

21. (canceled)

22. The method of claim 1, wherein the sample comprises one or more radical scavengers.

23. (canceled)

24. The method of claim 1, wherein the biological molecule comprises a protein.

25. (canceled)

26. The method of claim 1, further comprising identifying labeling of the biological molecule with the trifluoromethyl and hydroxyl and/or nitro radicals using mass spectrometry.

27.-41. (canceled)

42. A method of labeling a biological molecule with trifluoromethyl radicals, the method comprising:
a) providing a sample comprising a trifluoromethyl radical precursor and a biological molecule; and
b) generating a plasma in the sample, thereby converting the trifluoromethyl radical precursor into trifluoromethyl radicals which interact with the biological molecule, thereby labeling the biological molecule.

43. The method of claim 42, wherein the trifluoromethyl radical precursor is sodium triflinate.

44. The method of claim 42, wherein the biological molecule comprises a protein.

45. (canceled)

46. The method of claim 42, further comprising identifying labeling of the biological molecule using mass spectrometry.

47. A kit comprising one or more radical precursors, one or more buffers, and/or one or more radical quenchers.

48. The kit of claim 47, wherein the one or more radical precursors are selected from sodium triflinate and hydrogen peroxide, the one or more buffers are selected from PBS and HEPES buffer, and the one or more radical quenchers are selected from methionine, cysteine, homocysteine, or taurine.

49. The kit of claim 47, wherein the one or more radical precursors are selected from sodium triflinate and tert-butyl hydroperoxide (TBHP), the one or more buffers are selected from PBS and HEPES buffer, and the one or more radical quenchers are selected from methionine, cysteine, homocysteine, or taurine.
